(19) United States
(12) Patent Application Publication
(10) Pub. No.: US 2026/0294854 A1
(43) Pub. Date: Oct. 1, 2026

(54) BETA-HYDROXYBUTYRATE FOR THE DIETARY MANAGEMENT OF DEPRESSION

(71) Applicant: Laureate Institute for Brain Research, Tulsa, OK (US)

(72) Inventors: Martin PAULUS, Tulsa, OK (US); Elena GROSS, Basel (CH)

(21) Appl. No.: 19/578,701

(22) Filed: Mar. 25, 2026

Related U.S. Application Data
(60) Provisional application No. 63/777,446, filed on Mar. 25, 2025.

(51) Int. Cl. A61K 31/19; A61K 9/00; A61K 31/07; A61K 31/122; A61K 31/185; A61K 31/197; A61K 31/198; A61K 31/205; A61K 31/355; A61K 31/375; A61K 31/4188; A61K 31/4415; A61K 31/455; A61K 31/51; A61K 31/519; A61K 31/525; A61K 31/593; A61K 31/714; A61K 33/00; A61K 33/04; A61K 33/06; A61K 33/14; A61K 33/18; A61K 33/24; A61K 33/30; A61K 33/32; A61K 45/06; A61P 25/24

(52) U.S. Cl. CPC A61K 31/19; A61K 9/0095; A61K 31/07; A61K 31/122; A61K 31/185; A61K 31/197; A61K 31/198; A61K 31/205; A61K 31/355; A61K 31/375; A61K 31/4188; A61K 31/4415; A61K 31/455; A61K 31/51; A61K 31/519; A61K 31/525; A61K 31/593; A61K 31/714; A61K 33/00; A61K 33/04; A61K 33/06; A61K 33/14; A61K 33/18; A61K 33/24; A61K 33/30; A61K 33/32; A61K 45/06; A61P 25/24

(57) ABSTRACT

Methods for the dietary management of depression in a subject in need thereof include administering a composition including a therapeutically effective amount of β-hydroxybutyrate (βHB), salt thereof, or a metabolic precursor thereof to the subject. The composition of βHB, salt thereof, or the metabolic precursor thereof may be formulated as food, a food product, a food supplement, or a combination thereof. The composition may be formulated for oral administration, optionally together with micronutrients. Administration elevates circulating βHB and reduces depressive symptoms as assessed by standardized clinical rating scales.

### CROSS REFERENCE TO RELATED APPLICATIONS

This application claims priority from U.S. Provisional Application No. 63/777,446, filed on Mar. 25, 2025 in the U.S. Patent and Trademark Office, the disclosure of which is incorporated herein by reference in its entirety.

### TECHNICAL FIELD

The present disclosure relates generally to the use of exogenous ketone bodies such as β-hydroxybutyrate (βHB), or a metabolic precursor thereof, for the dietary management of depression.

### BACKGROUND

Depressive disorders, including major depressive disorder (MDD), are among the most prevalent and disabling medical conditions worldwide, contributing substantially to impaired functioning and reduced quality of life across the lifespan. Global public health estimates indicate that depression affects hundreds of millions of individuals and remains a leading contributor to years lived with disability (1).

Current standards of care rely primarily on psychotherapy and monoaminergic pharmacotherapies (e.g., selective serotonin reuptake inhibitors (SSRIs), serotonin-norepinephrine reuptake inhibitors (SNRIs), tricyclic antidepressants, monoamine oxidase inhibitors, and related agents). While many patients benefit, a clinically consequential fraction fails to achieve sustained remission after first-line interventions. In large effectiveness studies that more closely reflect real-world practice, remission after an initial antidepressant trial occurs in only a subset of patients, and the probability of remission decreases across additional sequential treatment steps (2).

A core limitation of prevailing treatment paradigms is that depression is biologically heterogeneous. Convergent evidence supports that, in at least a subset of patients, depressive symptoms co-occur with systemic inflammation, oxidative stress, and mitochondrial/bioenergetic impairments rather than reflecting a purely monoaminergic disturbance (3). Consistent with the clinical relevance of this heterogeneity, higher baseline C-reactive protein (CRP)—a marker of systemic inflammation—has been associated with smaller symptom reductions during SSRI monotherapy in at least some clinical datasets, suggesting that inflammatory phenotypes may be less responsive to conventional monoaminergic strategies (4).

These observations motivate interventions that directly target energy metabolism and immunometabolic signaling in a manner that is practical for sustained use. Nutritional ketosis induced by a ketogenic diet (KD)—a high-fat, very low-carbohydrate dietary intervention that increases circulating ketone bodies—has recently shown an efficacy signal in a randomized clinical trial in treatment-resistant depression (TRD), with greater improvement after 6 weeks in the KD group compared with a control diet group (5). However, sustained adherence to strict carbohydrate restriction is often difficult in real-world settings, limiting feasibility as a scalable intervention (6).

Exogenous ketone supplementation provides an alternative means to increase circulating ketone bodies without requiring persistent dietary restriction. Human metabolic studies demonstrate that exogenous ketone drinks can increase blood β-hydroxybutyrate (βHB), but the magnitude and kinetics of exposure depend on formulation and feeding state (6). Moreover, commonly used ketone salt preparations may include substantial quantities of the L-βHB enantiomer, which differs from the physiologic D-βHB produced endogenously and exhibits different kinetic behavior in humans (6). Accordingly, there remains a need for well-characterized, orally administered ketone compositions that reliably deliver βHB in a form and dosing schedule compatible with longer-term use in depressive populations.

### SUMMARY

βHB is not solely an oxidative fuel; it is also a signaling metabolite with causal, experimentally demonstrated effects on pathways plausibly linked to depression subtypes characterized by inflammation and impaired bioenergetics. In mechanistic studies, βHB suppresses activation of the NLRP3 inflammasome and reduces downstream pro-inflammatory cytokine production (7). In additional mechanistic work, βHB has been shown to act as an endogenous inhibitor of class I histone deacetylases (HDACs), thereby linking metabolic state to transcriptional programs involved in oxidative stress resistance (8). These causal molecular effects provide a coherent biological rationale for evaluating βHB-based interventions in depression, particularly where inflammation and impaired bioenergetics appear to be relevant contributors.

A translational constraint is that downstream biological effects are likely to be determined not only by ketone exposure but also by the capacity of mitochondrial and redox systems to utilize ketone-derived substrates efficiently. This creates a rationale for compositions that combine βHB with complementary micronutrients and cofactors participating in oxidative phosphorylation and cellular redox metabolism, while maintaining an electrolyte profile suitable for repeated daily dosing. According to an exemplary embodiment of the present invention, in a representative, analytically characterized KetoSwiss formulation, a unit dose provides approximately 4.91 g βHB together with calcium (~417 mg) and magnesium (~275 mg), consistent with calcium/magnesium βHB salts, and further includes mitochondrial cofactors and bioactive vitamin forms such as ubiquinol (~49.6 mg), riboflavin-5′-phosphate (~15.6 mg), thiamine (~3.54 mg), pyridoxal-5′-phosphate (~2.48 mg), and L-carnitine (~125 mg), along with taurine (~165 mg) and antioxidant micronutrients (e.g., ascorbic acid ~60.6 mg; total tocopherols ~13.8 mg). Vitamin B3 is provided predominantly as niacinamide (~33.5 mg niacinamide equivalents per unit dose), a form that supports NAD metabolism and does not activate the HCAR2/GPR109A receptor, distinguishing NAD-centric metabolic support from receptor agonism as the primary plausible B3-linked mechanism in such a composition (9).

Taken together, these considerations support the need for compositions and methods that enable reliable delivery of βHB, optionally in combination with mitochondrial and redox-supportive micronutrients, as a practical approach for the dietary management of depression and related depressive symptomatology.

Chronic inflammation is a recognized contributor to the pathogenesis of many neurological conditions, including Alzheimer's disease, Parkinson's disease, and MS. The KD and similar diets have been shown to reduce inflammatory markers across these conditions, suggesting a personalized nutritional approach may be beneficial in managing neurological health (10). Additionally, gut-brain axis dysregulation, often linked to epilepsy and systemic inflammation, could potentially be ameliorated by KD interventions (11). Reductions in amyloid plaque deposition and the promotion of neuronal repair mechanisms further support the use of the KD in managing neurodegenerative disorders (12).

The KD has demonstrated considerable promise in some studies as a therapeutic tool in a variety of neurological, metabolic, and psychiatric conditions. The evidence suggests significant metabolic and neurobiological effects to the KD, with broad clinical applicability ranging from diabetes to neurodegenerative and psychiatric disorders, from the KD. However, careful patient selection and monitoring are essential due to potential risks and variability in individual response. Further research is needed to fully elucidate the underlying mechanisms and to optimize the implementation of the KD alone in clinical practice. Moreover, while the KD has shown promise in neurological conditions like epilepsy, its strict dietary requirements limit its practicality for long-term management of any disease or condition, particularly one as complex as depression.

βHB, a key KB produced during fasting or ketogenic diets, exhibits a broad range of physiological effects with significant therapeutic potential. Recent research has demonstrated that βHB is not only an energy substrate but also functions as a regulator of various cellular processes across different tissues, including the brain, muscles, and mitochondria.

One of the notable properties of βHB is its role in regulating proteostasis, particularly in aging and neurodegenerative diseases. For instance, exogenous βHB has been found to ameliorate amyloid-β pathology in nematode models, and it facilitates the clearance of neurodegeneration-related proteins in the mouse brain (13). This makes βHB a promising candidate for developing therapies aimed at enhancing proteostasis in Alzheimer's disease and other neurodegenerative conditions. Additionally, βHB has demonstrated the ability to enhance mitochondrial function, which is crucial in various metabolic and neuroprotective roles. Specifically, βHB has been shown to improve mitochondrial respiration during conditions of increased energy demand, such as hyperexcitation in neurons. This enhancement is mediated in part by Ca2+ signaling and is independent of certain mitochondrial carriers (14). Moreover, βHB administration post-ischemia has been observed to improve mitochondrial complex I and II activities, suggesting its utility in recovery following ischemic events (15). The hermetic response induced by βHB, which involves the activation of nuclear factor erythroid 2-related factor 2 (Nrf2), sirtuins, and AMP-activated protein kinase (AMPK), further supports its role in promoting resistance to oxidative and inflammatory stress (16).

Based on animal experiments, there has been some suggestion that βHB might have antidepressant and anti-inflammatory properties, particularly in the context of neuroinflammation. Studies have shown that direct infusion of βHB into the prefrontal cortex reduces depressive behaviors and decreases inflammatory cytokine levels, such as TNF-α, in animal models (17). Chronic administration of βHB has also produced significant behavioral effects comparable to treatments like fluoxetine, and it has been shown to augment the effects of sub-therapeutic doses of antidepressants (18). These findings indicate that βHB could serve as an innovative treatment approach for mood disorders, particularly those involving inflammation (19). In one embodiment, the methods and compositions herein concurrently manage inflammation in the subject.

Furthermore, βHB has been demonstrated to exert anticatabolic effects on muscle protein kinetics, particularly during acute inflammation. It significantly reduces whole-body phenylalanine degradation and decreases protein breakdown, which highlights its potential utility in preserving muscle mass during catabolic states (20). From a molecular signaling perspective, βHB functions as both a signaling metabolite and an epigenetic regulator. It has been shown to inhibit class I histone deacetylases (HDACs), leading to increased histone acetylation and the activation of genes involved in oxidative stress resistance, such as Forkhead box class O3a (FOXO3A) and metallothionein 2 (MT2) (8). This epigenetic regulation is particularly relevant in conditions of oxidative stress, aging, and calorie restriction, indicating that βHB may have a broader role in promoting health span. Finally, the pharmacokinetic profile of βHB has been extensively characterized, revealing insights into its absorption, distribution, and clearance dynamics. The population pharmacokinetic (PK) model indicates that βHB absorption follows a complex pattern involving multiple consecutive first-order inputs, with significant effects of formulation and dosing on its bioavailability (21). This information is crucial for optimizing βHB administration in clinical settings.

Collectively, these findings highlight the diverse therapeutic potential of βHB in regulating cellular metabolism, protecting against oxidative stress, enhancing mitochondrial function, and modulating neuroinflammation. These properties make βHB a versatile candidate for the development of novel therapies targeting a wide range of conditions, including neurodegenerative diseases, mood disorders, and muscle wasting. Thus, it is believed that the administration of a therapeutically effective amount of βHB or a pharmaceutically acceptable metabolic precursor to a patient may treat, ameliorate, or otherwise help manage depression or the symptoms thereof in such patient as further described herein. Thus, the disclosure provides a solution to the need for additional therapies and management techniques to address depression and symptoms thereof.

### DETAILED DESCRIPTION

This description and any accompanying drawings that illustrate aspects, embodiments, implementations, or applications should not be taken as limiting—the claims define the protected invention. Various changes may be made without departing from the spirit and scope of this description and the claims.

In this description, specific details are set forth describing some embodiments consistent with the present disclosure. Numerous specific details are set forth in order to provide a thorough understanding of the embodiments. It will be apparent, however, to one of ordinary skill in the art that some embodiments may be practiced without some or all of these specific details. The specific embodiments disclosed herein are meant to be illustrative but not limiting. One of ordinary skill in the art may realize other elements that, although not specifically described here, are within the scope and the spirit of this disclosure. In addition, to avoid unnecessary repetition, one or more features shown and described in association with one embodiment may be incorporated into other embodiments unless specifically described otherwise or if the one or more features would make an embodiment non-functional.

The present invention is directed to the dietary management of depression with administration of a composition including exogenous KB, such as βHB or its precursors (i.e., ketogenic compounds) formulated as a food, food product, supplement, or any combination thereof. Without being bound by theory, it is believed that such administration may elevate blood ketone levels without the need for dietary restrictions.

In another aspect, the present disclosure describes the treatment of depression by administration of a composition including exogenous KB. As used herein, “treatment” (including grammatical variations thereof such as “treat” or “treating”) refers to clinical intervention in an attempt to alter the natural course of the individual being treated. Treatment also includes management of depression and the symptoms thereof. Desirable effects of treatment may include, but are not limited to, one or more of the following: prophylaxis, preventing occurrence or recurrence of depression or symptoms associated with depression, alleviation of symptoms, diminishment of any direct or indirect pathological consequences of the depression, decreasing the rate of depression progression, amelioration or palliation of the depression, improved prognosis and cure. Without being bound by theory, it is believed that management of depression according to the present disclosure supports healthy mood balance, promotes balanced mood, promotes mental wellness, helps maintain positive mood and emotional wellbeing, supports emotional resilience, and combinations thereof.

In particular, “treatment” refers to the alleviation or elimination of symptoms, the amelioration of depression, and/or preventing the recurrence of disease or symptoms associated with depression. The term “prevention” (including grammatical variations thereof such as “prevent” or “preventing”) refers in, particular, to prophylaxis, and/or preventing occurrence or recurrence of depression or symptoms associated with depression. Furthermore, the term “amelioration” (including grammatical variations thereof such as “ameliorate” and “ameliorating”) refers, in particular, to a decrease in the intensity, duration, and/or frequency of the symptoms of depression, and may also include the prevention of the aggravation of the symptoms and/or the prevention of the recurrence of aggravated symptoms.

KB, as used herein and in the context of the present invention, includes β-hydroxybutyric acid, β-hydroxybutyrate, and/or a pharmaceutically acceptable β-hydroxybutyrate salt, or a combination of any of the foregoing, which are collectively denoted herein as “βHB,” as well as, acetoacetic acid, acetoacetate, and/or a pharmaceutically acceptable acetoacetate salt, or a combination of any of the foregoing, which are collectively denoted herein as “AcAc” (see, e.g., CAS No. 541-50-4).

In a preferred embodiment, the KB comprises a pharmaceutically acceptable β-hydroxybutyrate salt. Furthermore, the KB, as used herein and in the context of the present invention, may comprise β-hydroxybutyric acid. Thus, the KB may comprise a pharmaceutically acceptable β-hydroxybutyrate salt or β-hydroxybutyric acid, or a combination thereof.

A pharmaceutically acceptable salt, as used herein, may be selected from the group consisting of: a potassium salt, a sodium salt, a calcium salt, a magnesium salt, an arginine salt, a zinc salt, a barium salt, an iron salt, a copper salt, a manganese salt, molybdenum salt, a lithium salt, a phosphor salt, a sulfur salt, an iodine salt, a selenium salt, a chromium salt, a cobalt salt, a lysine salt, a leucine salt, a histidine salt, an ornithine salt, a creatine salt, an agmatine salt, a citrulline salt, a methyl glucamine salt and a carnitine salt, or a combination of at least two of any of the foregoing salts.

Preferably, the pharmaceutically acceptable salt comprises at least 1, 2, 3 or 4, preferably all, salts selected from the group consisting of: a calcium salt, a sodium salt, a potassium salt, and a magnesium salt. For example, the pharmaceutically acceptable salt may be a blend of sodium, calcium, potassium, magnesium mineral salts at a mixing ratio of about 1:2:1:2, about 1:1:1:2 or about 1:1:1:1. In one embodiment, preferably no one salt is more than about 40% of the mixture. In an exemplary embodiment, the salt is a combination of calcium and magnesium mineral salts.

In one embodiment, it is preferred to use a combination of several salts. In particular, by increasing the number of different salts, the total tolerated dose may be increased. βHB, as used herein, further includes any enantiomer, i.e., D-βHB and/or L-βHB, and/or a racemate (i.e., D/L-βHB) of beta-hydroxybutyric acid and/or its conjugate base beta-hydroxybutyrate. Beta-hydroxybutyric acid is also known as β-hydroxybutyric acid or 3-hydroxybutyric acid, and beta-hydroxybutyrate is also known as or β-hydroxybutyrate or 3-hydroxybutyrate (CAS No. 300-85-6).

As used herein, the βHB may include D-βHB and/or L-βHB, the racemic DL-βHB or a substantially pure form of the isomer D-βHB. Preferably, the D-βHB contains greater than 50%, and at least about 60%, 70%, 80%, 90%, 95%, 99%, 99.9%, 99.99% or 100% of D-βHB. In a preferred form, “substantially pure” means at least about 90%, preferably at least about 95%, and more preferably about 99% of the specified enantiomer, in all cases relative to the other enantiomer. In some embodiments, the βHB contains more than 50%, and at least about 60%, 70%, 80%, 90%, 95%, 99%, 99.9%, 99.99% or 100% of L-βHB. In a preferred form, the L-βHB is substantially pure. However, as mentioned herein and in the context of the invention, βHB preferably refers to pure or substantially pure D-βHB.

D-βHB, also known as (R)-βHB, as used herein, refers to a specific enantiomer of βHB. The enantiomer can be produced in the liver by humans. Furthermore, D-βHB or a precursor of D-βHB was shown to increase the brain metabolic efficiency and regulate the transcriptional and the epigenetic state of cells; WO2010/021766A1 and Shimazu et al., Science. 2013 Jan. 11; 339(6116):211-4.

The formulae of β-hydroxybutyric acid (left), D-β-hydroxybutyric acid (middle) and D-β-hydroxybutyrate (right) are depicted in the following:

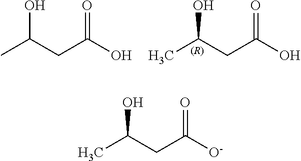

The formula of acetoacetic acid is depicted in the following:

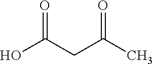

βHB and AcAc (the anion/salt form of the acetoacetate depicted above) are the major types of KB that are produced by humans in response to fasting or a ketogenic diet, and thus may also refer to “endogenous KB,” which may be measured, e.g., in the blood of a subject as described herein. In particular, βHB and AcAc are produced by the liver from fatty acids released from adipose tissue in times of starvation, fasting, glucose and/or carbohydrate deprivation and/or prolonged intense exercise. They can be used as an alternative energy substrate to glucose by most tissues of the body, most notably the brain, which cannot metabolize any other energy substrate apart from glucose, lactate and KB.

However, βHB and AcAc may also refer to “exogenous KB” when they are administered to a subject, as described herein and in the context of the invention, although they may be chemically identical to the “endogenous KB.”

The terms “KB,” “ketogenic compound,” “βHB,” and “ketone ester”, as used herein and in the context of the present invention, do not include the following, which are considered non-ketogenic compounds: poly-3-hydroxybutyrate, poly-D-(−)-3-hydroxybutryic acid, a co-polymer of poly-3-hydroxybutyrate such as poly(β-malic acid)-b-poly(β-hydroxybutyrate) and/or other polyesters, particularly those that comprise at least 10000, 5000, 1000, 500, 200, 100, 50, 20, or 10 units of monomeric βHB and that are not effectively metabolized into monomeric βHB in the body and/or that are used for the production of plastic such as biodegradable plastic, e.g. poly(3-hydroxybutyrate-co-3-hydroxyvalerate), or poly(3-hydroxybutyrate-co-4-hydroxybutyrate). In particular, the inventive composition provided herein preferably does not include (by volume and/or weight) more than about 90%, 70%, 50%, 30%, 10%, or more preferably not more than about 1%, or not more than about 0% poly-3-hydroxybutyrate, poly-D-(−)-3-hydroxybutyric acid, a co-polymer of poly-3-hydroxybutyrate such as poly(β-malic acid)-b-poly(β-hydroxybutyrate). In one embodiment, the invention is substantially or entirely free of the foregoing and/or other comparable non-ketogenic polyesters.

In particular, as used herein and in the context of the present invention, the term “ketogenic compound” refers to a precursor of βHB and/or AcAc. In particular, a ketogenic compound, as used herein, is a specific chemical compound or a defined mix of chemical compounds. In particular, the term “ketogenic compound”, as used herein, is not intended to reference a naturally occurring composition or food to which no specific chemical compound has been added as disclosed herein.

A precursor of an endogenous KB, i.e., βHB and/or AcAc, produces βHB and/or AcAc upon administration to a subject or during preparation of the precursor for administration to a subject. Preferably herein, the precursor of βHB and/or AcAc is a metabolic precursor of βHB and/or AcAc. In particular, a metabolic precursor is metabolized in the body of a subject as described herein into βHB and/or AcAc. For example, a metabolic precursor, when administered to a human or animal body, is metabolized, e.g., in the liver, to produce D-βHB and AcAc, preferably in a physiologically acceptable ratio.

Furthermore, the ketogenic compound may comprise a compound comprising an acetoacetyl- and/or 3-hydroxybutyrate moiety.

Preferably herein and in the context of the present invention, the ketogenic compound comprises a ketone ester, or a pharmaceutically acceptable salt thereof, preferably a ketone ester.

An ester is a chemical compound derived from an acid in which at least one —OH hydroxyl group is replaced by an —O-alkyl (alkoxy) group as in the substitution reaction of a carboxylic acid and an alcohol. Thus, herein, an ester formed by the union of two compounds means that these two compounds are esterified, in particular via an acid group of one compound and an alcohol group of the other compound.

The term “ketone ester” herein, refers to any ester that is a precursor, preferably a metabolic precursor, of βHB and/or AcAc, as described herein. Thus, a ketone ester may comprise, in particular, a 3-hydroxybutyrate (βHB) moiety, an acetoacetyl moiety, and/or at least one moiety that itself is a precursor of βHB and/or AcAc such as a 1,3-butanediol moiety or a middle chain fatty acid moiety.

Thus, in the context of the present invention, the ketone ester may comprise an ester of βHB and/or AcAc.

Thus, the ketone ester may comprise an ester formed by the union of βHB with βHB, βHB with AcAc, or AcAc with AcAc, e.g., 3-hydroxybutyl-(R)-3-hydroxybutyrate.

Furthermore, the ketone ester may comprise an ester formed by the union of βHB or AcAc with a monohydric, dihydric or trihydric alcohol. For example, the ketone ester may comprise an ester formed by the union of βHB with 1,3-butanediol or glycerol, wherein the 1,3-butanediol or glycerol may be further esterified with a fatty acid, e.g., a medium chain fatty acid. Thus, the ketone ester may comprise, for example, an ester formed by the union of βHB or AcAc with 1,3-butanediol, for example, an (R)-3-hydroxybutyrate-R-1,3-butanediol monoester. In a further example, the ketone ester may comprise an ester formed by the union of βHB or AcAc with glycerol, for example, glyceryl tris(3-hydroxybutyrate. The glycerol may be further esterified with at least one fatty acid, e.g., a medium chain fatty acid.

The ketone ester may also comprise an ester formed by the union of βHB or AcAc (or both, but in one embodiment preferably βHB) with a fatty acid, e.g., a medium chain fatty acid.

Furthermore, the ketone ester may comprise an ester formed by the union of 1,3-butanediol with at least one fatty acid, e.g., a medium chain fatty acid, for example, 1,3-butanediol esterified with two caproic acids.

Preferably, the ketone ester is an ester of D-βHB and/or the ketone ester is metabolized in the body of a subject into D-βHB and/or acetoacetate, preferably at least D-βHB.

Furthermore, the ketogenic compound may comprise an amide of βHB and/or AcAc, or both, or a pharmaceutically acceptable salt thereof.

Furthermore, the ketogenic compound may comprise a compound selected from the group consisting of: D-beta-hydroxybutyrate-D-1,3-butanediol; (3R)-hydroxybutyl-(3R)-hydroxybutyrate; acetoacetyl-1,3-butanediol; acetoacetyl-R-3-hydroxybutyrate; acetoacetylglycerol; 3-hydroxybutyl 3-hydroxybutanoate; (3-hydroxy-1-methyl-propyl) 3-hydroxybutanoate; 3-(3-hydroxybutanoyloxy)butyl 3-hydroxybutanoate; 3-(3-hydroxybutanoyloxy)butanoic acid; 3-hydroxybutyl 3-oxobutanoate; 3-hydroxy-1-methyl-propyl) 3-oxobutanoate; 3-(3-oxobutanoyloxy)butyl 3-oxobutanoate; 3-(3-oxobutanoyloxy)butanoic acid; 2,3-dihydroxypropyl 3-oxobutanoate; [2-hydroxy-1-(hydroxymethyl)ethyl] 3-oxobutanoate; [2-hydroxy-3-(3-oxobutanoyloxy)propyl] 3-oxobutanoate; [3-hydroxy-2-(3-oxobutanoyloxy)propyl] 3-oxobutanoate; 2,3-bis(3-oxobutanoyloxy)propyl 3-oxobutanoate; any one of the formulae

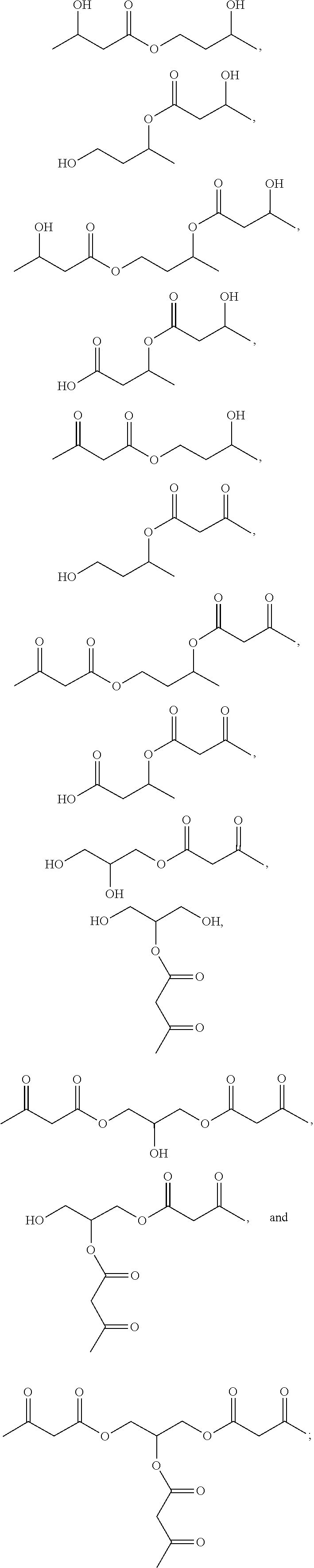

a combination of any of the foregoing; and a pharmaceutically acceptable salt of any of the foregoing.

Herein and in the context of the present invention, the ketogenic compound may comprise a metabolic precursor of βHB and/or AcAc selected from the group consisting of: 1,3-butanediol, triacetin, a fatty acid such as a medium chain fatty acid, or a compound comprising a fatty acid such as a medium chain fatty acid, e.g., a medium chain triglyceride, or a ketogenic amino acid such as leucine or lysine; or a pharmaceutically acceptable salt of said metabolic precursor. In particular, 1,3-butanediol refers to CAS No. 107 88 0, and triacetin refers to CAS No. 102-76-1.

In particular herein and in the context of the present invention, the term “fatty acid” includes short chain fatty acids, medium chain fatty acids, and long chain fatty acids as described herein, e.g. acids with aliphatic tails of 4 to 21 carbon atoms, i.e. 4, 5, 6, 7, 8, 9, 10, 11, 12, 13, 14, 15, 16, 17, 18, 19, 20, or 21 carbon atoms, e.g., 4, 5, 6, 7 or 8 carbon atoms, preferably 6 or 8, carbon atoms, more preferably 8 carbon atoms. Preferably herein, said chain (aliphatic tail) is saturated.

In particular herein in the context of the present invention, the terms “medium chain fatty acid”, “middle chain fatty acid” or “MCFA”, refer to an aliphatic monocarboxylic acid comprising a chain of 6, 7, 8, 9, 10, 11 or 12 carbon atoms, preferably 6, 7, or 8 carbon atoms. In one embodiment, the medium chain fatty acid includes 8 carbon atoms. Thus, the MCFA include preferably caproic acid, caprylic acid, capric acid and lauric acid, preferably caprylic acid or capric acid, more preferably caprylic acid. However, in the context of the present invention, also other fatty acids can be used in place of a medium chain fatty acid, e.g. long chain fatty acids with aliphatic tails of 13 to 21 carbons, or short chain fatty acids with 4 or 5 carbon atoms i.e., as long as they can be metabolized into βHB and/or AcAc. In particular, short chain fatty acids with 4 or 5 carbon atoms are very ketogenic and thus can be well used instead of medium chain fatty acids herein and in the context of the present invention.

The term “medium chain triglyceride”, “middle chain triglyceride” or “MCT”, as used herein, refer to an ester that is formed by the union of glycerol (see, e.g., CAS No. 56-81-5) and three MCFA. Furthermore, herein and in the context of the present invention, a MCT may be replaced by an ester that is formed by the union of glycerol and three short-, middle- or long-chain fatty acids, or a combination thereof.

As used herein, the term “ketogenic amino acid” refers to an amino acid that can be degraded to Acetyl-CoA, a precursor of endogenous KB. Leucine and lysine are ketogenic amino acids that are exclusively ketogenic. Isoleucine, phenylalanine, tryptophan and tyrosine are ketogenic amino acids that are also glucogenic. Thus, the ketogenic amino acid may comprise, in particular, leucine lysine, isoleucine, phenylalanine, tryptophan and/or tyrosine, or a combination thereof.

In one embodiment, the KB comprises D-β-hydroxybutyrate and/or D-β-hydroxybutyric acid, and/or the ketogenic compound is metabolized into D-β-hydroxybutyrate and/or D-β-hydroxybutyric acid. It is also preferred herein and in the context of this embodiment, the KB does not comprise L-β-hydroxybutyrate and/or L-β-hydroxybutyric acid, and/or said ketogenic compound is not metabolized into L-β-hydroxybutyrate and/or L-β-hydroxybutyric acid.

The methods of the present invention include treating depression or nutritionally treating depression via dietary management, such as reducing the symptoms or severity and/or frequency of one or more symptoms of depression or the depression itself and/or supporting metabolic dysfunction, in a patient or subject in need thereof, by administering a therapeutically effective amount of a composition including a KB including βHB or a pharmaceutically acceptable precursor (e.g., a ketogenic compound) as described herein thereof to the patient or subject. Symptoms of depression typically include a persistent sad, anxious, or “empty” mood; feelings of hopelessness or pessimism; feelings of irritability, frustration, or restlessness; feelings of guilt, worthlessness, or helplessness; loss of interest or pleasure in hobbies and activities; fatigue, lack of energy, or feeling slowed down; difficulty concentrating, remembering, or making decisions; difficulty sleeping, including waking too early or oversleeping; changes in appetite or unplanned weight changes; physical aches or pains, headaches, cramps, or digestive problems without a clear physical cause that do not go away with treatment; thoughts of death or suicide or suicide attempts; increased anger or irritability; becoming withdrawn, negative, or detached; increased engagement in high-risk activities; greater than normal impulsivity; increased use of alcohol or drugs; isolating from family and friends; inability to meet work or family responsibilities, or ignoring other important roles; problems with sexual desire and performance; or a combination thereof.

In some embodiments, a symptom of depression is measured according to a diary assessment, an assessment by clinician or caregiver, a clinical rating scale, or by functional magnetic resonance imaging (fMRI). In one or more embodiments, the fMRI measures the amygdala blood oxygen level-dependent (BOLD) response in the patient or subject.

In various embodiments, a symptom of depression is measured using a clinical depression rating scale, such as a Quick Inventory of Depressive Symptomatology (QIDS)-16 scale, a QIDS-16 daily scale, a Hamilton Depression Rating scale, a Beck Depression Inventory scale, a Montgomery-Asberg Depression Rating Scale (MADRS), a Clinical Global Impression Scale, a Zung Self-Rating Depression Scale, a Raskin Depression Rating Scale, and/or a Young Mania Rating Scale, or any other suitable reproducible scale used by those of ordinary skill in the art.

In several embodiments, a symptom of depression is measured using a Spielberger's Trait and Anxiety Inventory, a Generalized Anxiety Disorder 7-Item Scale, a Warwick-Edinburgh Mental Wellbeing Scale, a Flourishing Scale, a Snaith Hamilton Anhedonia Pleasure Scale, a Life Orientation Test, a Meaning in Life Questionnaire, a Brief Resilience Scale, a Dysfunctional Attitudes Scale, a 44-item Big Five Inventory, a Peters 21-item Delusional Inventory, an Examination of Anomalous Self-Experience, a Ruminative Responses Scale, a White Bear Suppression Inventory, a Barrett Impulsivity Scale, a Brief Experiential Avoidance Questionnaire, a Modified Tellegen Absorption Questionnaire, a Scale to Assess Therapeutic Relationship, a Credibility/Expectancy Questionnaire, a Connectedness to Nature Scale, a Political Perspective Questionnaire, a Social Connectedness Scale, a Bech-Rafaelsen Mania Rating Scale, a Revised Santa Clara brief compassion scale, a Gratitude Questionnaire, a Short Suggestibility Scale, a Rosenberg Self-Esteem Scale, a Universality Subscale of the Spiritual Transcendence Scale, an Oxford Questionnaire on the Emotional Side-effects of Antidepressants, a Lauks Emotional Intensity Scale, a Sexual Dysfunction Questionnaire, a Brief Index of Sexual Functioning for Women, a Sexual Perceptions Questionnaire, a Barnes Akathisia Rating Scale, a Work Productivity and Activity Impairment Questionnaire, a Work and Social Adjustment Scale, a Connectedness Questionnaire, a Standard Assessment of Personality, a Positive and Negative Syndrome Scale, a Mastery Insight Scale, a Self-Reflection and Insight Scale, a Psychological Insight Scale, a Metaphysical Beliefs Questionnaire, a Spiritual Bypassing Scale, an Adverse Childhood Experience Questionnaire, a Therapeutic Music Experience Questionnaire, a Setting Questionnaire, an Absorption in Music Scale, a Psychedelic Predictor Scale, a Surrender Scale, a EuroQOL-5 Dimension-3 Level Scale, a Columbia-Suicide Severity Rating Scale, a Suicidal Ideation Attributes Scale, or any combination(s) thereof.

Without being bound by theory, it is believed that βHB (and its precursor(s)) may be effective in treating depression (including reducing the symptoms of depression) due to the (1) ability to enhance energy metabolism and mitochondrial function, (2) anti-inflammatory and immunomodulatory effects, (3) regulation of proteostasis, (4) reduction of oxidative stress, and/or (5) enhancement of neurogenesis and synaptic plasticity.

Depression is linked to impaired brain energy metabolism and mitochondrial dysfunction in regions like the prefrontal cortex and hippocampus. βHB (and its precursor(s)) acts as an efficient energy substrate, bypassing glycolysis to fuel mitochondrial adenosine triphosphate (ATP) production directly. The βHB or precursor generally stabilizes mitochondrial membranes, reduces reactive oxygen species (ROS), and improves oxidative phosphorylation efficiency as described herein. By enhancing mitochondrial function, βHB can restore energy homeostasis, supporting synaptic plasticity and cognitive function. In one or more embodiments, the depression that is treated with βHB (or precursor) is metabolically-impaired depression, where patients have metabolic dysfunctions, such as insulin resistance.

Chronic inflammation is believed to contribute to depression, particularly in conventional treatment-resistant depression (TRD) cases. Elevated cytokines may disrupt neurotransmitter systems. βHB inhibits the NOD-like receptor protein 3 (NLRP3) inflammasome, reducing pro-inflammatory cytokines such as interleukin-1 beta (IL-1β) and tumor necrosis factor alpha (TNF-α). Suppressing inflammation via βHB may alleviate neurotoxicity and promote neurogenesis. In some embodiments, the depression that may advantageously be treated with βHB is inflammation-associated depression, particularly where patients have elevated inflammatory markers.

Accumulation of damaged proteins may contribute to synaptic and neuronal damage in depression. βHB enhances the clearance of misfolded proteins, protecting against proteotoxic stress. βHB's promotion of proteostasis may advantageously maintain neural circuit integrity.

Oxidative stress damages lipids, proteins, and DNA, exacerbating neuronal dysfunction in depression. Mitigating oxidative stress preserves neuronal viability. βHB may advantageously decrease mitochondrial ROS production and aids in detoxifying free radicals.

Reduced neurogenesis and impaired synaptic plasticity are features of depression. Enhancing these processes may improve mood regulation and cognitive symptoms. βHB may advantageously improve synaptic plasticity, long-term potentiation, and dendritic branching. In certain embodiments, the depression that is treated with βHB is neurodegeneration-linked depression, where patients experience depression and neurodegenerative disorders.

In some embodiments, βHB is administered as an exogenous supplement selected from a βHB salt, βHB ester, or a combination thereof. Thus, unless otherwise specified, the term “βHB” as used herein is intended to refer to βHB or any salt, ester, or combination of the foregoing. In various embodiments, βHB is administered as a metabolic precursor that is selected from acetoacetate; 1,3-butanediol; a medium-chain triglyceride that metabolizes to βHB; or a combination thereof.

In one or more embodiments, a composition including a KB or ketogenic compound as described herein is administered as part of food, a food product, or a supplement for the dietary management of depression. For example, the composition may be formulated as a powdered product that can be mixed into or deposited onto food, formed into or reconstituted into a beverage and/or consumed directly. The terms “food” and “food product” are generally used interchangeably herein and refer to any substance consumed to provide nutritional support for an organism. Any discussion throughout this disclosure of a pharmaceutical composition or a pharmaceutically acceptable excipient or acceptable carrier may, when referring to a preferred embodiment involving the dietary management of depression, be prepared as such a supplement or included within a food or food product. Food may be of biological origin, and contain essential nutrients, such as carbohydrates, fats, proteins, vitamins, or minerals. In one embodiment, the food may consist essentially of an aqueous solution or suspension. Food is ingested by a subject and assimilated by the subject's cells to provide energy, maintain life, or stimulate growth. A food product can be taken in by a subject or administered to a subject. Preferably, a food product is for eating as a solid (which may contain some liquid) or drinking as a liquid (which may contain some dissolved or undissolved solid component(s), e.g., boba tea or a slurry or other form of liquid).

A “food supplement,” as used herein, refers to a concentrated source of nutrients with a nutritional and/or physiological effect. In the present invention, the main nutrient comprised in a food supplement may be the KB (e.g., βHB) and/or ketogenic compound described herein. A food supplement may further comprise other nutrients, in particular vitamins or minerals. Otherwise, a food supplement may be taken in by a subject or administered, or in solid, liquid, semi-solid or other form, all as above for a food or food product.

In some embodiments, the inventive composition may be provided as a solid or powder form. Solid or powder compositions may include one or more additional components configured to lower the hygroscopicity of the composition. For example, various anticaking agents, flow agents, and/or moisture absorbers, in types and amounts that are safe for consumption, may be included. Such additional components may include one or more of an aluminosilicate, ferrocyanide, carbonate or bicarbonate salt, silicate (e.g., sodium or calcium silicate), phosphate salt (e.g., tricalcium phosphate), talcum, powdered cellulose, and the like. Any suitable amount of such foregoing additional components may be selected by one of ordinary skill in the art based on the guidance in the art and the disclosure herein.

In alternative embodiments, the inventive compositions may be provided as a liquid, such as in the form of a shot or mouth spray for fast delivery and absorption, or as a gel. Liquid or gel forms may include one or more carriers, such as water, ethanol, glycerin, propylene glycol, 1,3-propandiol, and the like, into which the beta-hydroxybutyrate/acetoacetate compounds are dissolved or dispersed. The composition may include flavoring agents that help mask the somewhat poor taste of beta-hydroxybutyrate compounds. These flavoring agents may include essential oils, such as peppermint, natural and artificial sweeteners, and other flavorants known in the art. Any suitable amount of such foregoing carriers, flavoring agents, or other pharmaceutically acceptable carriers or excipients may be selected by one of ordinary skill in the art based on the guidance in the art and the disclosure herein.

Furthermore, the inventive composition may be formulated as medical food and/or food for special medical purposes. A medical food and/or food for special medical purposes may be also considered a food product, food supplement or nutritional aid as described herein.

Furthermore, a food for special medical purposes, as used herein, refers to food products for the dietary management (under medical supervision), of individuals who suffer from certain diseases, disorders or medical conditions. These foods are intended for the exclusive or partial feeding of people whose nutritional requirements cannot be met (or not fully met) by normal foods.

Furthermore, a medical food, as used herein, refers to a food formulated to be consumed or administered enterally under the supervision of a physician or other medical practitioner, and which is intended for the specific dietary management of a disease or condition for which distinctive nutritional requirements, based on recognized scientific principles, are established by medical evaluation.

A food for special medical purpose and/or a medical food relates, in particular, to the therapeutic uses of the invention.

In some embodiments, the inventive composition includes a KB and/or ketogenic compound as described herein formulated as a pharmaceutical composition and/or a medicament. In other embodiments, the inventive composition includes a KB and/or ketogenic compound as described herein and is formulated as a food product or supplement. In yet other embodiments, the invention composition co-administers pharmaceutical composition and/or a medicament along with a food product or supplement, or the pharmaceutical composition and/or medicament is administered in combination with a food product or supplement. Thus, the inventive composition provided herein may be used for treatment as described herein.

As an alternative to formulation as a food product or supplement, the inventive composition may be formulated as a pharmaceutical composition. In practical use, the KB and/or ketogenic compound may be combined as the active ingredients in intimate admixture with a pharmaceutically acceptable carrier according to conventional pharmaceutical compounding techniques. The carrier may take a wide variety of forms and comprises a number of components depending on the form of preparation desired for administration. The inventive compositions include, but are not limited to, one or more suspensions, solutions and elixirs; aerosols; or excipients, including, but not limited to, starches, sugars, microcrystalline cellulose, diluents, granulating agents, lubricants, binders, disintegrating agents, and the like; or any combination of the foregoing. As to any of the pharmaceutically acceptable carriers or excipients disclosed herein, each may be generally included with the compositions herein in a suitable amount based on the guidance in the art and the disclosure herein.

In one or more embodiments, the KB and/or ketogenic compound is formulated into a composition that may comprise a (1:1) mix of D-β-hydroxybutyric acid and a pharmaceutically acceptable salt mix of D-β-hydroxybutyrate, and optionally further a desiccant.

Compositions of the KB and/or ketogenic compound described herein may be administered in a variety of dosage forms, depending on its intended use, as is well known in the art, based on the guidance herein. In one or more embodiments, the KB and/or ketogenic compound described herein is administered as an oral dosage form or an intravenous dosage form. When an oral dosage form is administered, it may for example be formulated as a powder (e.g., for a reconstitutable liquid), tablet (including orally disintegrating, for example), pill, capsule, caplet, gelcap, or liquid solution.

In solid dosage forms for oral administration, the composition is mixed with one or more pharmaceutically acceptable carriers and/or any of the following: (1) fillers or extenders, such as starches, lactose, sucrose, glucose, mannitol, and/or silicic acid; (2) binders, such as carboxymethylcellulose, alginates, gelatin, polyvinyl pyrrolidone, sucrose and/or acacia; (3) disintegrating agents, such as agar-agar, calcium carbonate, potato or tapioca starch, alginic acid, certain silicates, and sodium carbonate; (4) lubricants, such as talc, calcium stearate, magnesium stearate, solid polyethylene glycols, sodium lauryl sulfate, and mixtures thereof; (5) glidants, such as silicon dioxide and talc; (6) antioxidants, such as ascorbic acid, alpha tocopherol, ascorbyl palmitate, ascorbates, and isoascorbates; and (7) coloring agents, such as Red 40, Red 3, Yellow 5, and Yellow 6. Solid compositions may be used as fillers in soft and hard-filled gelatin capsules using such excipients as lactose or milk sugars, as well as high molecular weight polyethylene glycols.

A tablet may be made by compression or molding, optionally with one or more accessory ingredients. Compressed tablets may be prepared using binder (for example, gelatin or hydroxypropylmethyl cellulose), lubricant, inert diluent, preservative, disintegrant (for example, sodium starch glycolate or cross-linked sodium carboxymethyl cellulose), and a surface-active or a dispersing agent. Molded tablets may be made by molding in a suitable machine a mixture of the composition moistened with an inert liquid diluent. Tablets, and other solid dosage forms, such as dragees, capsules, pills, and granules, may optionally be scored or prepared with coatings and shells, such as enteric coatings and other coatings well known in the pharmaceutical-formulating art to facilitate patient compliance, improve the taste, provide for sustained or delayed release, or the like.

Liquid dosage forms for oral administration include pharmaceutically acceptable emulsions, microemulsions, solutions, suspensions, syrups, and elixirs. In addition to the composition, the liquid dosage forms may contain inert diluents commonly used in the art, such as, for example, water and one or more other solvent(s), solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, cyclodextrins and mixtures thereof.

The inventive compositions may also be formulated for parenteral administration by injection (subcutaneous, bolus injection, intramuscular, or intravenous), and may be dispensed in a unit dosage form, such as a multidose container or an ampule. Such compositions for parenteral administration may be in the form of suspensions, solutions, emulsions, or the like in aqueous or oily vehicles, and in addition to the active ingredients may contain one or more formulary agents, such as dispersing agents, suspending agents, stabilizing agents, preservatives, and the like.

Formulations suitable for intravenous administration include the KB and/or ketogenic compound described herein combined with a pharmaceutically acceptable carrier, such as sterile water or sterile isotonic saline. Such formulations may be prepared, packaged, or sold in a form suitable for bolus administration or for continuous administration. Injectable formulations may be prepared, packaged, or sold in unit dosage form, such as in ampules or in multidose containers typically containing a preservative. Formulations for intravenous administration include, but are not limited to, suspensions, solutions, emulsions in oily or aqueous vehicles, pastes, and implantable sustained-release or biodegradable formulations. Such formulations may further include one or more additional ingredients including, but not limited to, suspending, stabilizing, or dispersing agents.

The intravenous compositions may be prepared, packaged, or sold in the form of a sterile injectable aqueous or oily suspension or solution. This suspension or solution may be formulated according to the known art, and may include, in addition to the KB and/or ketogenic compound described herein, additional ingredients such as one or more dispersing agents, wetting agents, or suspending agents. Such sterile injectable formulations may be prepared using a non-toxic acceptable diluent or solvent, such as water or 1,3-butanediol, for example. Other acceptable diluents and solvents include, but are not limited to, Ringer's solution, isotonic sodium chloride solution, and fixed oils such as synthetic mono- or di-glycerides.

Furthermore, the invention relates to a method for treating and/or preventing depression in a subject which comprises administering to the subject an effective amount of the inventive combination or composition provided herein. In one or more embodiments, the compositions may be administered in single or divided doses, from one to four times a day.

The terms “disorder”, “disease” and “medical condition” are used herein interchangeably and may refer to a pathophysiological response to external or internal factors, a disruption of normal or regular functions in the body or a part of the body and/or an abnormal state of health that interferes with the usual activities or feeling of wellbeing.

As used herein, “about” means within ±10% of a cited value or range, and in preferred embodiments may refer to values of ±5%, ±2%, and ±1% of a cited value. The term “about” generally refers to a range of numbers that one of ordinary skill in the art would consider literally the same as the cited value, and would typically consider similar values even outside these ranges as being equivalent (e.g., having the same function or result) unless otherwise specified.

### Modes of Administration

The present disclosure provides a composition that can be administered through multiple nutritional or ingestion-related modalities, thereby enhancing patient adherence, broadening consumer applicability, and enabling tailored delivery based on user preference or physiological need. The composition is formulated to maintain its stability, bioavailability, and functional performance across a range of administration conditions, including: (1) administration concurrently with food; (2) sequential administration relative to food intake; and (3) incorporation directly into a food or beverage product.

In certain embodiments, the composition is administered concurrently with food. Co-administration may enhance tolerability, improve gastrointestinal comfort, or modulate release characteristics. The composition may be ingested simultaneously with a meal, snack, or beverage without compromising its stability or efficacy. The formulation may optionally include excipients that facilitate dispersion, dissolution, or interaction with dietary components.

The feeding state of the subject at the time of administration may affect the absorption kinetics of βHB. In some embodiments, the composition is administered in a fed state, which may moderate the rate of βHB absorption and provide a more gradual elevation of blood ketone levels. In other embodiments, the composition is administered in a fasted state, which may result in more rapid absorption and a higher peak blood βHB concentration. The timing of administration relative to meals may be adjusted based on individual tolerance and preference.

In additional embodiments, the composition is administered sequentially with respect to food intake. Sequential administration includes delivery of the composition either before or after consumption of food within a defined temporal window. This approach may be used to optimize absorption kinetics, influence metabolic pathways, or coordinate the composition's activity with physiological states associated with feeding. The timing interval may vary depending on the intended effect, and the composition remains effective across such timing variations.

In further embodiments, the composition is incorporated directly into a food or beverage matrix to create a functional food product. The composition may be blended, infused, encapsulated, or otherwise integrated into solid, semi-solid, or liquid food forms, including but not limited to bars, baked goods, dairy products, confectionery items, nutritional beverages, or powdered drink mixes. When incorporated into a food product, the composition retains its functional properties and remains stable under typical processing, storage, and consumption conditions. This mode of administration enables seamless integration into daily dietary routines and supports consumer-friendly delivery formats.

In several embodiments, the composition is administered as a standalone supplement between meals. For example, the composition may be consumed mid-morning, mid-afternoon, or in the evening, independent of meal times. In one embodiment consistent with the three-times-daily dosing regimen described herein, the composition is administered in the morning, at noon, and in the evening, where one or more of these doses may be taken with food or between meals depending on individual preference.

The composition may be consumed with or without additional food, and the timing relative to meals may be adjusted based on individual tolerance, preference, and desired pharmacokinetic profile. In some cases, administration with food may improve tolerability, while in other cases, administration in a fasted state may be preferred to achieve higher peak ketone levels. The flexibility in administration timing allows the composition to be integrated into a variety of dietary patterns and lifestyles.

The ability to administer the composition through any of the above modes provides significant flexibility and broadens its applicability across diverse user populations. This multi-modal approach allows customization of delivery based on dietary habits, lifestyle preferences, physiological considerations, or therapeutic objectives. The composition's stability and performance across these modes represent one important aspect of the invention.

### Administration to Subjects Exhibiting Elevated CRP Levels

In certain embodiments, the invention provides methods for administering the composition to subjects exhibiting CRP level greater than 1 mg/L, as further described in Example 1. CRP is a circulating biomarker of systemic inflammation that may be measured in blood or serum using standard clinical assays. Elevated CRP (greater than 1 mg/L) has been associated with inflammatory phenotypes of depression that may be less responsive to conventional monoaminergic antidepressants such as SSRIs. Based on the disclosure herein, this association creates a rationale for interventions that target immunometabolic pathways rather than, or in addition to, monoaminergic signaling.

In some embodiments, the subject has a baseline CRP greater than 1 mg/L. In other embodiments, the subject has a baseline CRP greater than 2 mg/L. In still other embodiments, the subject has a baseline CRP greater than 3 mg/L. In one embodiment, the subject has a baseline CRP between about 1 mg/L and about 10 mg/L. In some embodiments, the methods and compositions herein concurrently manage depression and inflammation in the subject.

Without being bound by theory, βHB may be particularly suited for subjects with elevated CRP because βHB inhibits the NLRP3 inflammasome and reduces downstream pro-inflammatory cytokines such as IL-1β and TNF-α. By addressing an inflammatory component that may contribute to depressive symptomatology, βHB-containing compositions may provide benefit in subjects for whom inflammation is a relevant pathophysiological factor.

Subjects with CRP >1 mg/L may be identified as having an elevated systemic inflammatory state relative to individuals with CRP at or below this threshold. Elevated CRP can serve as an indicator of underlying inflammatory activity, metabolic dysregulation, or heightened immune activation. Because the composition described herein is designed to modulate pathways associated with inflammation, immune signaling, or related physiological processes, administration to subjects meeting this biomarker criterion may enhance the likelihood of achieving a therapeutic, prophylactic, or physiological benefit.

In some embodiments, the method comprises selecting a subject whose CRP level exceeds 1 mg/L and administering to the subject an effective amount of the composition. The CRP level may be measured using any clinically accepted assay, including high-sensitivity CRP assays. The threshold of CRP >1 mg/L may be used alone or in combination with additional biomarkers, clinical features, or risk factors to identify subjects most likely to respond to the composition.

In additional embodiments, subjects with CRP >1 mg/L may exhibit improved responsiveness, enhanced biomarker modulation, or greater reduction in inflammation-associated parameters following administration of the composition compared with subjects whose CRP levels are at or below 1 mg/L. The present invention further contemplates stratifying subjects based on CRP levels to tailor dosing regimens, treatment intervals, or combination-therapy strategies, provided such variations remain within the scope of the claimed methods.

The CRP-based selection criterion may be applied across a range of indications involving inflammatory or immune-related components, including but not limited to metabolic disorders, cardiovascular risk states, autoimmune conditions, or chronic inflammatory syndromes. In certain embodiments, the composition is administered as a monotherapy; in other embodiments, it is administered in conjunction with one or more additional agents that target complementary biological pathways.

In some embodiments, the method further includes measuring CRP in the subject prior to initiating administration of the composition. In other embodiments, the method includes monitoring CRP during the course of administration. In certain embodiments, a reduction in CRP during administration may indicate a favorable anti-inflammatory response.

The CRP threshold may be used as a selection criterion for identifying subjects who may benefit from dietary management with βHB-containing compositions. In some aspects, subjects with CRP greater than 1 mg/L may be selected for administration of the composition as a standalone intervention or as an adjunct to standard antidepressant therapy.

### Dosage and Testing of βHB

In the context of the present invention, the βHB or β-hydroxybutyrate salt may be included in the compositions herein at a dose of about 1 to about 100 g, more preferably about 5 to about 40 g. In one embodiment, the dose is 5 g. In some embodiments, the dose may be about 10 to about 20 g, and in one example it is 12 g, 15 g, or 18 g.

The dose or content of other KB or ketogenic compounds described herein can be readily adjusted to have a similar efficacy as the doses or contents of the βHB or β-hydroxybutyrate salt provided herein, such as by a medical practitioner of ordinary skill in the art and particularly based on the guidance provided herein.

In particular, administration of a composition including a KB and/or ketogenic compound provided herein in the context of the present invention to a subject increases the KB concentration, i.e., the βHB concentration, in the blood of the subject, preferably to between about 0.3 mM and about 5 mM. In one embodiment, the blood concentration of βHB is between about 0.5 mM and about 5 mM (e.g., 0.5 mM to 1 mM), while in another it is between about 1 mM and about 3 mM. In yet another embodiment, the blood concentration of βHB is between about 0.08 mM and about 0.5 mM.

In one embodiment, administration of about 15 g/day βHB (e.g., about 5 g per serving administered three times daily) yields measurable post-dose ketone exposure. For example, an 8-week administration regimen of a βHB-containing supplement produced median serum βHB values of approximately 0.15-0.16 mM at early post-dose timepoints and approximately 0.09 mM at a later post-dose timepoint at week 8, while placebo values remained lower, as further described in Example 1 below.

Furthermore, administration to a subject of a composition including a KB and/or ketogenic compound provided herein in the context of the present invention may increase the KB concentration, i.e., the βHB concentration, in the blood of the subject at least about 1.5-fold over a corresponding baseline concentration. In particular, the baseline concentration refers to the concentration measured before ingestion of a KB or ketogenic compound.

Thus, determining the KB concentration, i.e., βHB concentration, in the blood upon administration of a KB or ketogenic compound, further allows determination of the dose of a KB and/or ketogenic compound that should be used in the context of this inventive disclosure including in the inventive compositions provided herein. Hence, the βHB salts can be readily replaced with other KB or ketogenic compounds described herein without any particular difficulties, subject to potential dose modification as discussed above. However, one preferred KB or ketogenic compound to be used in the context of the present invention comprises βHB or otherwise as set forth herein.

Preferably, in the context of the present invention, the KB concentration in the blood is increased within about 2 h, about 1 h, about 30 min, or even about 15 min or less, preferably within about 30 min or less upon administration of the KB or ketogenic compound in any form described herein. Furthermore, the increased KB concentration in the blood may be maintained for at least about 1, about 2, about 4, about 6, about 8, about 10, about 12 or about 14 hours, preferably for at least about 1 to about 2 hours, more preferably for at least about 2 hours.

For example, the KB and/or ketogenic compound, e.g., the βHB, may be administered to the subject in doses between about 0.01 and about 1 g/kg body weight. For example, the KB and/or ketogenic compound, e.g., the βHB, may be administered to the subject once, twice, three times or four times per day.

For example, the daily dose of the KB and/or ketogenic compound, e.g., the βHB, to be administered may be about 0.05 g/kg to about 1 g/kg body weight (i.e., about 3.5-70 g/70 kg), about 0.1 g/kg to about 0.7 g/kg body weight (i.e., about 7-49 g/70 kg), or about 0.2 g/kg to about 0.4 g/kg body weight (i.e., about 14-28 g/70 kg). For example, the daily dose of the KB and/or ketogenic compound, e.g., the βHB, to be administered may be about 3.5 g to about 70 g, about 5 g to about 50 g, or about 10 g to about 40 g, for example about 10 g, or about 20 g.

### Possible Combinations

Furthermore, the KB and/or ketogenic compound described herein may be combined with the micronutrient component as described herein. The micronutrient component includes one or more vitamins, antioxidants, co-enzymes, minerals (including trace minerals) and/or other micronutrients, or nutraceuticals that have been shown to be mitochondrial function enhancing, or any combination of the foregoing. In particular, the KB and/or ketogenic compound described herein may be combined with at least 1, 2, 3, 4, 5, 6, 7, 8, 9, 10, 12, 15, or 20, different vitamins, co-enzymes, minerals, micronutrients, or nutraceuticals selected from: Vitamin A (e.g., as beta-carotene or vitamin A palmitate), Vitamin C (e.g., as calcium ascorbate, ascorbic acid, ascorbyl palmitate, magnesium ascorbate, niacinamide ascorbate, acerola extract), Vitamin D3 (e.g., as cholecalciferol) or D2, Vitamin E (e.g., as mixed tocopherols containing D-alpha tocopherol succinate, D-alpha tocopherol, or D-alpha tocopherol acetate), Vitamin K (as Vitamin K1 or Vitamin K2), Thiamine (vitamin B1) (e.g., as thiamine HCI), Riboflavin (vitamin B2) (e.g., as riboflavin, riboflavin 5′-phosphate), Niacin (e.g., as 61% niacinamide, 38% niacin, 1% niacinamide ascorbate), Vitamin B6 (e.g., as pyridoxal 5′-phosphate, pyridoxine HCI), Folate (e.g., as L-5-methyltetrahydrofolate calcium salt), Vitamin B12 (e.g., as methylcobalamin or adenosylcobalamin), Biotin, Pantothenic acid (e.g., as D-calcium pantothenate with 5 mg pantethine), Calcium (e.g., as dicalcium phosphate, Ca ascorbate, D-calcium pantothenate, Ca D-glucarate, or calcium R-βHB), Iodine (e.g., as potassium iodide), Magnesium (e.g., as magnesium oxide, citrate, arginate, glycinate, taurinate, or ascorbate, or magnesium R-βHB), Zinc (e.g., as zinc citrate, zinc gluconate, or zinc mono-L-methionine sulfate), Selenium (e.g., as sodium selenite, selenium L-methionine, or Se-methyl L-selenocysteine), Copper (e.g., as copper bisglycinate chelate), Manganese (e.g., as manganese citrate, manganese bisglycinate chelate, or gluconate), Chromium (e.g., as chromium nicotinate), Molybdenum (e.g., as molybdenum amino acid chelate), Sodium (e.g., as sodium chloride), Potassium (e.g., as potassium citrate), CoQ10 (e.g., ubichinon or ubiquinol), L-Lysine, L-Leucine, Broccoli concentrate, N-acetyl-L-cysteine (NAC), Decaffeinated green tea extract (leaf), Acerola Extract, Inositol, Bitter orange citrus bioflavonoids, Fruit/berry blend (e.g., European elder, blackberry, blueberry, sweet cherry, cranberry, plum, persimmon (Diospyros kaki) powders), Taurine, Wild blueberry anthocyanin extract, Ashwagandha extract (root, leaf), Silymarin (from milk thistle extract (seed)), L-carnitine, (acetyl-) L-carnitine, L-carnitine tartrate, Trimethylglycine, fermented L-lysine, fermented L-leucine, citicoline sodium, sour cherry (tart cherry) proanthocyanidin powder (skin), pomegranate extract (fruit), Natural mixed tocopherols (providing gamma, delta, alpha, beta tocopherols), bilberry extract (fruit), grape proanthocyanidin extract (whole grape), grape seed proanthocyanidin extract, Quercetin, Bromelain, Lutein, Olive extract (fruit), Sesame seed extract, Luteolin, Apigenin, Boron (e.g., as boron amino acid chelate or calcium fructoborate), Lycopene (e.g., natural tomato extract), choline, betaine (trimethylglycine), SAMe (S-adenosylmethionine), uridine, iron, creatine, pyruvate, malate, succinate, fumarate, alpha-ketoglutarate, citric acid, glutathione, alpha-lipoic acid, melatonin, astaxanthin, PQQ, resveratrol, nicotinamide riboside, nicotinamide mononucleotide, ribose, allulose, lactoferrin, colostrum, phosphatidylcholine, phosphatidylserine, omega-3 fatty acids (DHA/EPA), urolithin A, methylene blue, sulforaphane, ergothioneine, spermidine, betaine (TMG), uteolin, rutin, hesperidin, curcumin, black currant extract, lithium, lycopene, lutein, zeaxanthin, beta-carotene, tocotrienols, mixed carotenoids, chlorophyll, spirulina, chlorella, mushroom extracts (reishi, lion's mane, cordyceps), probiotics (Lactobacillus, Bifidobacterium), prebiotics (inulin, FOS, GOS), resistant starch, myo-inositol, alanine, arginine, asparagine, aspartic acid, cysteine, glutamic acid, glutamine, glycine, histidine, isoleucine, leucine, lysine, methionine, phenylalanine, proline, serine, threonine, tryptophan, tyrosine, and valine, or any combination thereof. In some embodiments, the micronutrient component includes one or more vitamins, antioxidants, co-enzymes, minerals (or trace minerals), micronutrients, or nutraceuticals described above are or include a mitochondrial cofactor.

The amounts of micronutrient component, e.g., antioxidants, vitamins, coenzymes, minerals (or trace minerals), or combination thereof administered as a compositions or in any suitable pharmaceutical form with the βHB component (e.g., salt, acid, metabolic form, etc.) may vary depending on the intended use, the form of the composition, and the nutritional needs of the subject. The following ranges are provided on a per-unit-dose basis and may be scaled proportionally for compositions providing more or less than about 5 g of βHB per unit dose. One preferred example of a unit dose of the micronutrient component is as stick pack for reconstitution in water, although other suitable unit dose forms may be used, preferably those for oral ingestion, such as a liquid suspension or solution, or a solid or semi-solid edible form (e.g., a capsule, tablet, gummy, gel, water, etc.).

In some embodiments, the composition includes coenzyme Q10 (e.g., as ubiquinol) in an amount of about 5 mg to about 200 mg per unit dose, such as about 25 mg to about 100 mg per unit dose or about 40 mg to about 60 mg per unit dose, for example about 50 mg per unit dose.

In one or more embodiments, the composition includes riboflavin (vitamin B2) (e.g., as riboflavin-5′-phosphate) in an amount of about 1 mg to about 100 mg per unit dose, such as about 5 mg to about 50 mg per unit dose or about 10 mg to about 25 mg per unit dose, for example about 15 mg per unit dose.

In certain embodiments, the composition includes thiamine (vitamin B1) in an amount of about 0.5 mg to about 50 mg per unit dose, such as about 1 mg to about 10 mg per unit dose or about 3 mg to about 5 mg per unit dose, for example about 4 mg per unit dose.

In several embodiments, the composition includes vitamin B6 (e.g., as pyridoxal-5′-phosphate) in an amount of about 0.5 mg to about 50 mg per unit dose, such as about 1 mg to about 10 mg per unit dose or about 2 mg to about 5 mg per unit dose, for example about 3 mg per unit dose.

In certain embodiments, the composition includes niacin (e.g., as niacinamide) in an amount of about 5 mg to about 200 mg per unit dose, such as about 15 mg to about 75 mg per unit dose or about 20 mg to about 40 mg per unit dose, for example about 35 mg per unit dose.

In some embodiments, the composition includes L-carnitine in an amount of about 10 mg to about 500 mg per unit dose, such as about 50 mg to about 250 mg per unit dose or about 100 mg to about 150 mg per unit dose, for example about 125 mg per unit dose.

In one or more embodiments, the composition includes taurine in an amount of about 10 mg to about 1000 mg per unit dose, such as about 50 mg to about 500 mg per unit dose or about 100 mg to about 200 mg per unit dose, for example about 165 mg per unit dose.

In various embodiments, the composition includes vitamin C (e.g., as ascorbic acid) in an amount of about 10 mg to about 500 mg per unit dose, such as about 30 mg to about 150 mg per unit dose or about 50 mg to about 100 mg per unit dose, for example about 60 mg per unit dose.

In several embodiments, the composition includes vitamin E (e.g., as mixed tocopherols) in an amount of about 1 mg to about 100 mg per unit dose, such as about 5 mg to about 30 mg per unit dose or about 10 mg to about 25 mg per unit dose, for example about 14 mg per unit dose.

In some embodiments, the composition includes calcium (e.g., from calcium βHB salt) in an amount of about 50 mg to about 1000 mg per unit dose, such as about 200 mg to about 600 mg per unit dose or about 300 mg to about 500 mg per unit dose, for example about 417 mg per unit dose.

In certain embodiments, the composition includes magnesium (e.g., from magnesium βHB salt) in an amount of about 50 mg to about 800 mg per unit dose, such as about 100 mg to about 500 mg per unit dose or about 200 mg to about 400 mg per unit dose, for example about 275 mg per unit dose.

When the composition is administered in multiple daily doses (e.g., three times daily), the total daily intake of each micronutrient may be approximately three times the per-unit-dose amount. For example, administration of three unit doses per day may provide a total daily intake of about 15 g βHB, about 150 mg ubiquinol, about 47 mg riboflavin-5′-phosphate, about 10.6 mg thiamine, about 7.4 mg pyridoxal-5′-phosphate, about 100 mg niacinamide, about 375 mg L-carnitine, about 495 mg taurine, about 182 mg ascorbic acid, and about 41 mg mixed tocopherols.

The amounts may be adjusted by one of ordinary skill in the art based on the guidance herein and the nutritional needs of the subject. In some embodiments, the amounts are selected to provide between about 25% and about 300% of the recommended dietary allowance (RDA) or adequate intake (AI) for the respective micronutrient, where such values are established.

In one or more embodiments, the composition includes at least two, at least three, at least four, at least five, or at least six of the foregoing micronutrients. In one exemplary embodiment, the composition includes coenzyme Q10, riboflavin, thiamine, vitamin B6, niacin, L-carnitine, and taurine.

In one or more embodiments, the micronutrient components include one or more of vitamin B6, thiamine, riboflavin, niacin, coenzyme Q10, or L-carnitine, or any combination thereof. In certain embodiments, micronutrient components are selected from the group consisting of vitamin B6, thiamine, riboflavin, niacin, coenzyme Q10, and L-carnitine.

In some embodiments, the micronutrient component includes at least one of one or more vitamin(s), one or more mineral nutrient(s), one or more amino acid(s) or derivative(s) thereof, one or more antioxidant(s), or a combination thereof. In certain embodiments, the micronutrient component includes at least two of one or more vitamin(s), one or more mineral nutrient(s), one or more amino acid(s) or derivative(s) thereof, one or more antioxidant(s), or a combination thereof. In several embodiments, the micronutrient component includes at least three of one or more vitamin(s), one or more mineral nutrient(s), one or more amino acid(s) or derivative(s) thereof, one or more antioxidant(s), or a combination thereof. In various embodiments, the micronutrient component includes at least four of one or more vitamin(s), one or more mineral nutrient(s), one or more amino acid(s) or derivative(s) thereof, one or more antioxidant(s), or a combination thereof. In one or more embodiments, the micronutrient component includes at least five of one or more vitamin(s), one or more mineral nutrient(s), one or more amino acid(s) or derivative(s) thereof, one or more antioxidant(s), or a combination thereof. In other embodiments, the micronutrient component includes at least six of one or more vitamin(s), one or more mineral nutrient(s), one or more amino acid(s) or derivative(s) thereof, one or more antioxidant(s), or a combination thereof.

In some embodiments, the one or more vitamin(s) include one or more of vitamin A, vitamin C, vitamin D, vitamin E, vitamin K, vitamin B1, vitamin B2, vitamin B3, vitamin B5, vitamin B6, vitamin B7, vitamin B9, or vitamin B12. In various embodiments, the one or more mineral nutrient(s) include one or more of calcium, iodine, magnesium, zinc, selenium, manganese, chromium, molybdenum, sodium, or potassium. In several embodiments, the one or more amino acid(s) or derivative(s) thereof include one or more of lysine or leucine. In certain embodiments, the one or more antioxidant(s) comprise one or more of carnitine, taurine, or coenzyme Q10.

Thus, the inventive compositions provided herein, e.g., prepared as a food, a food product, food supplement or nutritional aid, may further comprise at least 1, 2, 3, 4, 5, 6, 7, 8, 9, 10, 12, 15, 20, of said vitamins, co-enzymes, minerals, micronutrients, or nutraceuticals or any combination of the foregoing categories. Advantageously, administration of βHB results in elevated blood ketone levels in a patient or subject without causing ketoacidosis. Ketoacidosis is a life-threatening condition when ketones build up in the blood and make the blood acidic.

Moreover, supplementation with the KB and/or ketogenic compound described herein is well tolerated in clinical trials for other conditions. Side effects are minimal and manageable. In several embodiments, the present methods include regular monitoring of ketone levels and metabolic parameters to ensure safety of treatment. In one or more embodiments, the methods herein include assessing mood, cognitive function, and a metabolic parameter of the patient or subject to evaluate the treating of depression, the reduction of symptoms of depression, or both.

In some embodiments, the KB and/or ketogenic compound described herein is administered alone in a therapeutically effective amount. The therapeutically effective amount varies based on patient-specific factors such as age, weight, sex, genetics, and underlying medical conditions. The amount administered to a patient or subject should elevate blood ketone levels to a therapeutic range without inducing ketoacidosis. Generally, the therapeutically effective amount of βHB or the precursor lies within the range of about 0.1-100 grams per day (e.g., 3-50 grams). This can be taken in one dose or divided doses daily. In an exemplary embodiment, the therapeutically effective amount of βHB or the precursor thereof is sufficient to elevate blood ketone levels to about 0.05 mM-5 mM. In one embodiment, the therapeutically effective amount of βHB or the precursor thereof is sufficient to elevate blood ketone levels to about 0.08 mM-0.3 mM post dose.

In other embodiments, the KB and/or ketogenic compound described herein is used in combination with a standard antidepressant, such as a selective serotonin reuptake inhibitor (SSRI), a serotonin-norepinephrine reuptake inhibitor (SNRI), an atypical antidepressant, a tricyclic antidepressant, or a monoamine oxidase inhibitor (MAOI). In various embodiments, the standard antidepressant is administered prior to administration of βHB, on the same day as the administration of βHB, or after administration of βHB.

Therapeutically effective dosage levels and dosage regimens for standard antidepressants may be readily determined by one of ordinary skill in the art, and may vary in connection with the dosage of any co-administered standard antidepressant. For example, therapeutic dosage amounts and regimens for pharmaceutical agents approved for sale are publicly available, for example as listed on packaging labels, in standard dosage guidelines, in standard dosage references such as the Physician's Desk Reference or other sources. In some embodiments, the therapeutically effective amount of the standard antidepressant, when administered in combination with the KB and/or ketogenic compound described herein is the same dosage amount that is used when the standard antidepressant is administered alone. In other embodiments, the therapeutically effective amount of the standard antidepressant, when administered in combination with the KB and/or ketogenic compound described herein, is a reduced dosage amount from the amount that is used when the standard antidepressant is administered alone. In various embodiments, the KB and/or ketogenic compound described herein and the standard antidepressant have a combined effect that is synergistic, which may indicate a lower dose of one or both when co-administered.

In some embodiments, the KB and/or ketogenic compound described herein is used in combination with a neuroprotective agent. In one or more embodiments, the neuroprotective agent is a ketogenic amino acid. A ketogenic amino acid is metabolized only to acetyl-CoA. In one or more embodiments, the ketogenic amino acid is administered in an amount of about 5 g to about 30 g.

In various embodiments, the administration of the KB and/or ketogenic compound described herein thereof is administered in combination with a low-carbohydrate diet. A low carbohydrate diet is a diet that limits the consumption of carbohydrates, such as those found in grains, starchy vegetables, and fruit. A daily limit of 0.7 to 2 ounces (20 to 57 grams) of carbohydrates is typical of a low-carbohydrate diet. As used herein, a “low-carbohydrate diet” means that less than about 25%, less than about 20%, or less than about 15%, of daily calories are derived from carbohydrates.

In certain embodiments, the KB and/or ketogenic compound described herein is used in combination with psychotherapy. Psychotherapy is used to treat mental health by talking with a psychologist, psychiatrist, or other mental health provider. It is also known as talk therapy, counseling, or more generally, therapy (including, for example cognitive therapy, cognitive behavioral therapy, etc.).

Administered “in combination,” as used herein, means that two (or more) different therapeutic agents or therapeutic activities are delivered to the patient or subject during the course of the patient's affliction with the disorder, e.g., the two or more therapeutic agents are delivered after the subject has been diagnosed with the disorder and before the disorder has been cured or eliminated or treatment has ceased for other reasons. In some embodiments, the delivery of one therapeutic agent is still occurring when the delivery of the second begins, so that there is overlap in terms of administration. This is sometimes referred to herein as “simultaneous” or “concurrent delivery” or in parallel. In other embodiments, the delivery of one therapeutic agent ends before the delivery of the other treatment begins, i.e., the therapeutic agents are administered sequentially in time. In some embodiments of either case, the therapeutic agents are synergistically more effective because of combined administration, e.g., a food-based composition herein is eaten shortly before or after a pharmaceutical composition herein is administered. Typically, administration of the composition might be within 1 minute to 4 hours of eating the food-based composition, although in other embodiments, this might be within 5 minutes to 2 hours or within 15 minutes to 1 hour.

The administration of the KB and/or ketogenic compound described herein offers a novel therapeutic approach for the treatment of depression and related conditions, and symptoms associated therewith. By targeting key pathophysiological pathways—including energy metabolism, inflammation, oxidative stress, and proteostasis—βHB provides a multifaceted mechanism of action. The present invention presents a promising alternative or adjunct to existing antidepressant therapies, especially for patients with TRD.

In some embodiments, a method of treating depression or reducing the symptoms of depression in a subject in need thereof, which includes administering a therapeutically effective amount of β-hydroxybutyrate (βHB), a salt thereof, or a pharmaceutically acceptable metabolic precursor thereof to the subject is described herein. The therapeutically effective amount of βHB or the pharmaceutically acceptable metabolic precursor thereof is between about 1 g to about 100 g. The βHB is generally selected to include a salt of βHB, an ester of βHB, or a combination thereof. The pharmaceutically acceptable metabolic precursor of βHB is typically selected to include acetoacetate; 1,3-butanediol; a medium-chain triglyceride that metabolizes to βHB; or a combination thereof.

The symptoms can include: a persistent sad, anxious, or “empty” mood; feelings of hopelessness or pessimism; feelings of irritability, frustration, or restlessness; feelings of guilt, worthlessness, or helplessness; loss of interest or pleasure in hobbies and activities; fatigue, lack of energy, or feeling slowed down; difficulty concentrating, remembering, or making decisions; difficulty sleeping, including waking too early or oversleeping; changes in appetite or unplanned weight changes; physical aches or pains, headaches, cramps, or digestive problems without a clear physical cause that do not go away with treatment; thoughts of death or suicide or suicide attempts; increased anger or irritability; becoming withdrawn, negative, or detached; increased engagement in high-risk activities; greater than normal impulsivity; increased use of alcohol or drugs; isolating from family and friends; inability to meet work or family responsibilities, or ignoring other important roles; problems with sexual desire and performance; or a combination thereof.

In various embodiments, the method further includes elevating a blood ketone level of the subject to a therapeutic level without causing ketoacidosis. In one embodiment, the method further includes reducing neuroinflammation in the subject. In other embodiments, the method further includes enhancing neurogenesis and synaptic plasticity in the brain of the subject. In still other embodiments, the method further includes improving mitochondrial function and energy metabolism in neuronal cells of the subject. In certain embodiments, the method further includes administering a low-carbohydrate diet to the subject. In several embodiments, the method further includes administering psychotherapy to the subject in conjunction with the administering of the βHB or the pharmaceutically acceptable metabolic precursor thereof.

In certain embodiments, the method further includes administering a therapeutically effective amount of an antidepressant agent to the subject. The antidepressant agent may be selected to include a selective serotonin reuptake inhibitor (SSRI), a serotonin-norepinephrine reuptake inhibitor (SNRI), an atypical antidepressant, a tricyclic antidepressant, or a monoamine oxidase inhibitor (MAOI), or any combination thereof. In other embodiments, the method further includes administering a therapeutically effective amount of an anti-inflammatory agent to the subject. The anti-inflammatory agent may be selected to include any suitable anti-inflammatory tolerated by the subject, which may include, for example, one or more corticosteroids (e.g., prednisone, hydrocortisone), NSAIDs (e.g., ibuprofen, naproxen, aspirin, celecoxib, diclofenac (e.g., as diclofenac sodium 1% gel), meloxicam, indomethacin, etc), or any combination of the foregoing. A suitable therapeutically effective amount of anti-inflammatory may be determined by a clinician or others of ordinary skill in the art to be amounts typically administered to treat inflammation as a primary indication. In one preferred embodiment, the method includes administering therapeutically effective amounts of an antidepressant agent and an anti-inflammatory agent to the subject in association with either other, which may be concurrent or sequential.

In one or more embodiments, the βHB or the pharmaceutically acceptable metabolic precursor thereof is administered as an oral dosage form or an intravenous dosage form. The oral dosage form may be selected to include a powder, a tablet, a capsule, or a liquid solution.

In some embodiments, the method further includes administering a therapeutically effective amount of a neuroprotective agent, such as a ketogenic amino acid.

In several embodiments, the method further includes assessing mood, cognitive function, and a metabolic parameter of the subject to evaluate the treating of depression, the reduction of symptoms of depression, or both.

In certain embodiments, the present disclosure provides a method of treating depression in a human subject, including orally administering to the subject a composition including β-hydroxybutyrate (βHB), a salt thereof, or a metabolic precursor thereof in an amount effective to reduce one or more depressive symptoms. In some embodiments, the depression includes major depressive disorder (MDD). In various embodiments, the composition is administered as a food, food product, food supplement, medical food, or food for special medical purposes for dietary management of depression.

In one embodiment, the βHB includes D-β-hydroxybutyrate. In other embodiments, the βHB is provided as a pharmaceutically acceptable salt selected from sodium, potassium, calcium, magnesium, or a combination thereof.

In one or more embodiments, a single serving of the composition provides about 1 g to about 20 g of βHB, and in a preferred embodiment, the composition provides about 2.5 g to 7.5 g of βHB. For example, the single serving provides about 5 g of βHB.

In some embodiments, the composition is administered in multiple daily doses. In an exemplary embodiment, the composition is administered three times per day.

In various embodiments, the composition is administered for at least 4 weeks. In one specific embodiment, the composition is administered for about 8 weeks.

In several embodiments, administering the composition elevates blood βHB in the subject relative to baseline. In one or more embodiments, post-dose blood βHB is between about 0.05 mM and about 1 mM. In certain embodiment, post-dose blood βHB is between about 0.08 mM and about 0.5 mM or about 0.05 mM to about 0.5 mM.

In certain embodiments, the subject is unmedicated for depression at initiation of administration of the composition.

In one or more embodiments, the method further includes assessing depressive symptoms using a clinician-rated or self-reported depression scale. In an exemplary embodiment, the depression scale includes the Montgomery-Åsberg Depression Rating Scale (MADRS) and/or the Quick Inventory of Depressive Symptomatology (QIDS). In several embodiments, the method reduces a MADRS total score by at least 5 points after administration for about 8 weeks.

In certain embodiments, the composition further includes one or more micronutrients selected from vitamin B6, thiamine, riboflavin, niacin, magnesium, coenzyme Q10, and L-carnitine. In one or more embodiments, the composition includes D-β-hydroxybutyrate calcium salt and D-β-hydroxybutyrate magnesium salt.

In various embodiments, the composition is packaged as unit-dose stick packs configured to be dissolved in water prior to ingestion. In an exemplary embodiment, each stick pack includes about 9.25 g of total formulation and provides about 5 g βHB.

In one or more embodiments, the method further includes performing functional MRI to assess reward-related activation in a nucleus accumbens region of interest and/or self-referential activation in a medial prefrontal cortex region of interest.

In some embodiments, the depression includes metabolically impaired depression, inflammation-associated depression, or neurodegeneration-linked depression.

In several embodiments, the method further includes administering psychotherapy to the subject in conjunction with administering the composition.

In various embodiments, the method further includes administering an antidepressant agent to the subject in conjunction with administering the composition. The antidepressant agent may include a selective serotonin reuptake inhibitor (SSRI), a serotonin-norepinephrine reuptake inhibitor (SNRI), an atypical antidepressant, a tricyclic antidepressant, or a monoamine oxidase inhibitor (MAOI).

In some embodiments, the metabolic precursor of βHB includes a ketone ester, 1,3-butanediol, acetoacetate, a medium-chain triglyceride, a ketogenic amino acid, or a combination thereof.

In certain embodiments, the present disclosure further provides a composition for dietary management and/or treatment of depression, including: (a) β-hydroxybutyrate (βHB), a salt thereof, or a metabolic precursor thereof; and (b) one or more micronutrients selected from vitamin B6, thiamine, riboflavin, niacin, magnesium, coenzyme Q10, and L-carnitine, wherein the composition is configured for oral administration.

In one or more embodiments, the βHB includes D-β-hydroxybutyrate. In some embodiments, the βHB is provided as a mineral salt including calcium βHB and magnesium βHB.

In several embodiments, the composition provides about 1 g to about 20 g βHB per serving. In an exemplary embodiment, the composition provides about 5 g βHB per serving.

In various embodiments, the composition is packaged as unit-dose stick packs for reconstitution in water. For example, each stick pack includes about 9.25 g of total formulation.

In several embodiments, the present disclosure additionally provides a kit including the composition as described herein and instructions for administering the composition to a subject with depression. In some embodiments, the instructions direct administration of one serving one to three times daily for at least 4 weeks. For example, the instructions direct administration for about 8 weeks.

In one or more embodiments, the present disclosure also provides a method for dietary management of depression in a human subject, including administering a composition including a therapeutically effective amount of β-hydroxybutyrate (βHB), a salt thereof, or a metabolic precursor thereof to the subject, wherein the composition is formulated as a food product, food supplement, or medical food.

In certain embodiments, the therapeutically effective amount is between about 1 g and about 100 g per day.

In various embodiments, the method further includes elevating a blood ketone level of the subject without causing ketoacidosis. In some embodiments, the method further includes reducing neuroinflammation in the subject. In one or more embodiments, the method further includes improving mitochondrial function and energy metabolism in neuronal cells of the subject. In several embodiments, the method further includes enhancing neurogenesis and synaptic plasticity in the brain of the subject. In certain embodiments, the method further includes administering a low-carbohydrate diet to the subject.

In one exemplary embodiment, the present disclosure encompasses methods and compositions for the dietary management of depression (which in one preferred embodiment is MDD) in a subject in need thereof, which comprises orally administering a composition includes a therapeutically effective amount of β-hydroxybutyrate (βHB), a salt thereof, or a metabolic precursor thereof to the subject, and a micronutrient component or combination of such components in the amounts described herein. In one preferred embodiment, the βHB includes D-β-hydroxybutyrate, while in the same embodiment or others the βHB is provided as a calcium salt, a magnesium salt, or a combination thereof. In another embodiment, the βHB includes L-β-hydroxybutyrate, while in the same embodiment or others the βHB is provided as a calcium salt, a magnesium salt, a sodium salt, or a combination thereof.

In some embodiments, the subject in need of dietary management of depression has a deficiency of at least one part of the micronutrient component. In one embodiment, the deficiency is compensated for by the composition.

In another embodiment, present disclosure encompasses methods and compositions for the dietary management of major depressive disorder in a subject in need thereof, which comprises orally administering a composition includes a 0.1 grams to 20 grams of β-hydroxybutyrate (βHB) (or optionally 0.5 grams to 20 grams, or 1 gram to 20 grams, or 0.1 grams to 15 grams), a salt thereof, or a metabolic precursor thereof to the subject in 2-3 daily doses for 2 weeks to 12 weeks (preferably 4-8 weeks), and a micronutrient component or combination of such components as described herein. In one preferred embodiment, the βHB includes D-β-hydroxybutyrate, while in the same embodiment or others the βHB is provided as a calcium salt, a magnesium salt, or a combination thereof. In one embodiment, the methods and compositions reduce a clinician-rated depression score in the subject, where the clinician-rated depression score optionally includes a Montgomery-Åsberg Depression Rating Scale (MADRS) score, and wherein the MADRS score is optionally reduced by at least 5 points after 8 weeks of administration.

In these exemplary embodiments, a therapeutically effective amount of an antidepressant agent with a pharmaceutically acceptable carrier may be administered to the subject in connection with the methods and compositions. Optionally, such antidepressant agent may include a selective serotonin reuptake inhibitor (SSRI), a serotonin-norepinephrine reuptake inhibitor (SNRI), an atypical antidepressant, a tricyclic antidepressant, or a monoamine oxidase inhibitor (MAOI), or any combination thereof.

In a further embodiment, the methods and compositions relate to methods and compositions for the dietary management of depression (which in one preferred embodiment is MDD) in a subject in need thereof, which comprises orally administering two to four times daily a composition including 2 g to 10 g of β-hydroxybutyrate (βHB), a salt thereof, or a metabolic precursor thereof, as a calcium salt, a magnesium salt, or a combination thereof to the subject, and 1 mg to 1000 mg per unit dose of a micronutrient component or combination of such components, or such other preferred unit dose amount(s) based on preferred micronutrient component(s) described herein. Optionally, the micronutrient component includes at least one of vitamin B6, thiamine, riboflavin, niacin, coenzyme Q10, L-carnitine, or a combination thereof.

The Abstract at the end of this disclosure is provided to comply with 37 C.F.R. § 1.72(b) to allow a quick determination of the nature of the technical disclosure. It is submitted with the understanding that it will not be used to interpret or limit the scope or meaning of the claims.

### EXAMPLES

The following examples are provided to illustrate certain embodiments of the disclosure and are not intended to be limiting.

### Example 1: Randomized Controlled Trial of βHB Supplementation in Major Depressive Disorder

An investigator-initiated, single-site, phase 2a, randomized, double-blind, placebo-controlled, parallel-group study evaluated an oral βHB-containing supplement administered in adults with major depressive disorder (MDD) for 8 weeks. Eligible participants were adults aged 18 to 65 years with current MDD confirmed by Mini-International Neuropsychiatric Interview (MINI), Patient Health Questionnaire-9 (PHQ-9) score ≥10, CRP value >1, BMI 19 to 38 kg/m², and no current psychotropic medications. In an interim dataset, 22 randomized participants had baseline and week 8 outcomes available for symptom scales.

Participants underwent a 10.5 hour screening/baseline evaluation split over 2 days at week 0 including questionnaires, neuroimaging before and after administration of the oral βHB-containing supplement and blood draws, office visits at week 2 (1.5 hours), week 4 (3 hours), week 6 (0.5 hours), week 8 (6 hours), a follow-up visit at week 10 (1.5 hours), and three phone calls between visits (weeks 1 and 3) during which a brief clinical assessment was obtained (10 minutes each).

Participants received either a βHB-containing supplement or matched placebo for 8 weeks, administered orally as stick packs three times daily. Each packet contained 5 g βHB as D-β-hydroxybutyrate calcium salt monohydrate and D-β-hydroxybutyrate magnesium salt trihydrate, within a 9.25 g formulation plus micronutrients (vitamin B6, magnesium, coenzyme Q10, niacin, L-carnitine, riboflavin, and thiamine). The specific active ingredients (and amounts) of the supplement are provided below in Table 1. The supplement also included other components: malic acid, natural flavor, silicon oxide, and Stevia extract.

TABLE 1
ACTIVE INGREDIENTS OF THE SUPPLEMENT

Vitamin A (70 mcg as Vitamin A Palmitate, 30 mcg as
Beta Carotene) 100 mcg
Vitamin C (as Ascorbic Acid) 50 mg
Vitamin D (as Cholecalciferol) 25 mcg
Vitamin E (as D-alpha Tocopherol Acetate) 5 mg
Vitamin K (12.5 mcg as Vitamin K1, 12.5 mcg as
Vitamin K2) 25 mcg
Thiamin (as Thiamine HCl) 2.5 mg
Riboflavin (as Riboflavin 5′-Phosphate Sodium) 12.5 mg
Niacin (27.5 mg as Niacinamide, 2.5 mg as Niacin) 30 mg NE
Vitamin B6 (as Pyridoxal 5′- Phosphate) 2.5 mg
Folate (as Quatrefolic ® Glucosamine 5-MTHF) 83.5 mcg DFE
Vitamin B12 (as Methylcobalamin) 50 mcg
Biotin 25 mcg
Pantothenic Acid (as Calcium D- Pantothenate) 7.5 mg
Calcium (as Calcium R-βHB) 350 mg
Iodine (as Potassium Iodide) 25 mcg
Magnesium (as Magnesium R- βHB) 220 mg
Zinc (as Zinc Gluconate) 5 mg
Selenium (as Selenium L-Methionine) 25 mcg
Manganese (as Manganese Bisglycinate Chelate) 0.75 mg
Chromium (as Chromium Nicotinate) 25 mcg
Molybdenum (as Molybdenum Chelate) 10 mcg
Sodium (as Sodium Chloride) 140 mg
Potassium (as Tripotassium Citrate) 190 mg
Magnesium R-βHB 2500 mg
Calcium R-βHB 2500 mg
L-Carnitine Tartrate 150 mg
Taurine 150 mg
Coenzyme Q-10 75 mg
Fermented L-Lysine 50 mg
Fermented L-Leucine 50 mg
Mixed Tocopherols 15 mg
Citicoline Sodium 10 mg
Boron (as Calcium Fructoborate) 250 mcg

Participants dissolved one packet in water and ingested orally three times daily (morning, noon, evening) for 8 weeks. The placebo consisted of a dextrose-based formulation designed to match appearance and administration. On neuroimaging days, participants ingested two packs together to increase ketone exposure during scanning. The third daily dose was taken in the evening.

Serum βHB was assessed to characterize ketone exposure, including post-dose sampling at 0, 20, 40, and 80 minutes during in-person visits. At week 8, median post-dose βHB values were higher in the active supplement arm compared to placebo at multiple post-dose timepoints, while baseline values were similar.

Week 8 post-dose βHB concentrations were higher in the βHB group compared to the placebo group at post-dose time points (20 minutes: 0.15 mM vs 0.04 mM; 40 minutes: 0.16 mM vs 0.05 mM; 80 minutes: 0.090 mM vs 0.035 mM), consistent with the active supplement increasing circulating ketones as intended.

Neuroimaging endpoints included (i) reward anticipation BOLD signal within the nucleus accumbens during a Monetary Incentive Delay (MID) task (P5vP0 contrast) and (ii) self-referential BOLD signal within medial prefrontal cortex during an adjective judgment task (SELFvWORD contrast). Depressive symptom outcomes included clinician-rated MADRS and self-reported QIDS-SR16.

In the interim dataset (N=22), Bayesian linear mixed-effects models estimated between-arm differences at week 8 and between-arm differences in change from baseline to week 8. Differences are reported as placebo minus active supplement; thus, positive values for symptom scales indicate lower (improved) symptom severity in the active supplement arm.

TABLE 2
PATIENT RESULTS

| | | | Difference in change | |
|---|---|---|---|---|
| | Week 8 difference | | (baseline→week 8) | |
| | (placebo − active) | | (placebo − active) | |
| Outcome | [95% CrI] | | [95% CrI] | |
| Primary fMRI | −0.02 | (−0.17 to 0.12) | 0.00 | (−0.17 to 0.18) |
| (nucleus | | | | |
| accumbens MID P5vP0) | | | | |
| Secondary fMRI | 0.00 | (−0.18 to 0.18) | −0.09 | (−0.33 to 0.16) |
| (mPFC SELFvWORD) | | | | |
| MADRS | 7.17 | (2.23 to 12.0) | 7.12 | (1.50 to 12.7) |
| (clinician-rated) | | | | |
| QIDS-SR16 | 1.95 | (−0.94 to 4.92) | 1.89 | (−1.11 to 4.83) |
| (self-reported) | | | | |

In this interim analysis, the primary and secondary neuroimaging endpoints showed no evidence of between-group separation at week 8, whereas clinician-rated depressive symptoms (MADRS) showed a credible advantage favoring the active βHB supplement. Self-reported symptoms (QIDS-SR16) trended in the same direction but were less precise.

Clinician-rated depressive symptoms (MADRS) improved more with βHB supplementation. The βHB-placebo difference at week 8 on MADRS was 7.17 points (95% credible interval [CrI], 2.23 to 12.0) and the βHB-placebo difference in change was 7.12 points (95% CrI, 1.50 to 12.7). Self-reported symptoms (QIDS-SR16) were directionally consistent (week 8 difference: 1.95 [95% CrI, −0.94 to 4.92]; change difference: 1.89 [95% CrI, −1.11 to 4.83]). Adherence was high and similar between arms (median, 94.16%; IQR, 87.01%-99.39%).

### REFERENCES

1. “Depression.” World Health Organization, World Health Organization, 2023, https://www.who.int/news-room/fact-sheets/detail/depression.
2. Trivedi M H, Rush A J, Wisniewski S R, et al. Evaluation of outcomes with citalopram for depression using measurement-based care in STAR*D: implications for clinical practice. Am J Psychiatry. 2006; 163(1): 28-40. doi:10.1176/appi.ajp.163.1.283.
3. Larrea A, Sánchez-Sánchez L, Diez-Martin E, et al. Mitochondrial Metabolism in Major Depressive Disorder: From Early Diagnosis to Emerging Treatment Options. J Clin Med. 2024; 13(6): 1727. Published 2024 Mar. 17. doi:10.3390/jcm130617274.
4. Jha M K, Minhajuddin A, Gadad B S, et al. Can C-reactive protein inform antidepressant medication selection in depressed outpatients? Findings from the CO-MED trial. Psychoneuroendocrinology. 2017; 78:105-113. doi:10.1016/j.psyneuen.2017.01.023.
5. Gao M, Kirk M, Knight H, et al. A Ketogenic Diet for Treatment-Resistant Depression: A Randomized Clinical Trial. JAMA Psychiatry. Published online Feb. 4, 2026. doi:10.1001/jamapsychiatry.2025.4431.
6. Stubbs B J, Cox P J, Evans R D, et al. On the Metabolism of Exogenous Ketones in Humans. Front Physiol. 2017; 8:848. Published 2017 Oct. 30. doi:10.3389/fphys.2017.00848.
7. Youm Y H, Nguyen K Y, Grant R W, et al. The ketone metabolite β-hydroxybutyrate blocks NLRP3 inflammasome-mediated inflammatory disease. Nat Med. 2015; 21(3): 263-269. doi:10.1038/nm.3804.
8. Shimazu T, Hirschey M D, Newman J, et al. Suppression of oxidative stress by β-hydroxybutyrate, an endogenous histone deacetylase inhibitor. Science. 2013; 339(6116):211-214. doi:10.1126/science.1227166.
9. Horimatsu T, Blomkalns A L, Ogbi M, et al. Niacin protects against abdominal aortic aneurysm formation via GPR109A independent mechanisms: role of NAD+/nicotinamide. Cardiovasc Res. 2020; 116(14):2226-2238. doi:10.1093/cvr/cvz303.
10. Kurowska A, Ziemichod W, Herbet M, Piatkowska-Chmiel I. The Role of Diet as a Modulator of the Inflammatory Process in the Neurological Diseases. Nutrients. 2023; 15.
11. Mukhtar I. Unravelling the critical role of neuroinflammation in epilepsy-associated neuropsychiatric comorbidities: A review. Prog Neuropsychopharmacol Biol Psychiatry. 2024; 136:111135.
12. Mentzelou M, Dakanalis A, Vasios G K, Gialeli M, Papadopoulou S K, Giaginis C. The Relationship of Ketogenic Diet with Neurodegenerative and Psychiatric Diseases: A Scoping Review from Basic Research to Clinical Practice. Nutrients. 2023; 15.
13. Madhavan S S, Roa Diaz S, Peralta S, Nomura M, King C D, Ceyhan K E, Lin A, Bhaumik D, Foulger A C, Shah S, Blade T, Gray W, Chamoli M, Eap B, Panda O, Diaz D, Garcia T Y, Stubbs B J, Ulrich S M, Lithgow G J, Schilling B, Verdin E, Chaudhuri A R, Newman J C. beta-hydroxybutyrate is a metabolic regulator of proteostasis in the aged and Alzheimer disease brain. Cell Chem Biol. 2024.
14. Llorente-Folch I, Dussmann H, Watters O, Connolly N M C, Prehn J H M. Ketone body beta-hydroxybutyrate (BHB) preserves mitochondrial bioenergetics. Scientific reports. 2023; 13:19664.
15. Lehto A, Koch K, Barnstorf-Brandes J, Viel C, Fuchs M, Klein J. ss-Hydroxybutyrate Improves Mitochondrial Function After Transient Ischemia in the Mouse. Neurochem Res. 2022; 47:3241-3249.
16. Kolb H, Kempf K, Rohling M, Lenzen-Schulte M, Schloot N C, Martin S. Ketone bodies: from enemy to friend and guardian angel. BMC Med. 2021; 19:313.
17. Kajitani N, Iwata M, Miura A, Tsunetomi K, Yamanashi T, Matsuo R, Nishiguchi T, Fukuda S, Nagata M, Shibushita M, Yamauchi T, Pu S, Shirayama Y, Watanabe K, Kaneko K. Prefrontal cortex infusion of beta-hydroxybutyrate, an endogenous NLRP3 inflammasome inhibitor, produces antidepressant-like effects in a rodent model of depression.
18. Pan S, Hu P, You Q, Chen J, Wu J, Zhang Y, Cai Z, Ye T, Xu X, Chen Z, Tong L, Huang C, He H. Evaluation of the antidepressive property of beta-hydroxybutyrate in mice. Behav Pharmacol. 2020; 31:322-332.
19. Yamanashi T, Iwata M, Kamiya N, Tsunetomi K, Kajitani N, Wada N, Iitsuka T, Yamauchi T, Miura A, Pu S, Shirayama Y, Watanabe K, Duman R S, Kaneko K. Beta-hydroxybutyrate, an endogenic NLRP3 inflammasome inhibitor, attenuates stress-induced behavioral and inflammatory responses. Scientific reports. 2017; 7:7677.
20. Thomsen H H, Rittig N, Johannsen M, Moller A B, Jorgensen J O, Jessen N, Moller N. Effects of 3-hydroxybutyrate and free fatty acids on muscle protein kinetics and signaling during LPS-induced inflammation in humans: anticatabolic impact of ketone bodies. The American journal of clinical nutrition. 2018; 108:857-867.
21. Shivva V, Cox P J, Clarke K, Veech R L, Tucker I G, Duffull S B. The Population Pharmacokinetics of D-beta-hydroxybutyrate Following Administration of (R)-3-Hydroxybutyl (R)-3-Hydroxybutyrate. The AAPS journal. 2016; 18:678-688.

What is claimed is:

1. A method for the dietary management of depression in a subject in need thereof, which comprises orally administering a composition comprising a therapeutically effective amount of β-hydroxybutyrate (βHB), a salt or acid thereof, or a metabolic precursor thereof, to the subject, and a micronutrient component comprising one or more vitamin(s), one or more mineral nutrient(s), one or more amino acid(s) or derivative(s) thereof, one or more antioxidant(s), or a combination thereof.

2. The method of claim 1, wherein the one or more vitamin(s) comprise one or more of vitamin A, vitamin C, vitamin D, vitamin E, vitamin K, vitamin B1, vitamin B2, vitamin B3, vitamin B5, vitamin B6, vitamin B7, vitamin B9, or vitamin B12.

3. The method of claim 1, wherein the one or more mineral nutrient(s) comprise one or more of calcium, iodine, magnesium, zinc, selenium, manganese, chromium, molybdenum, sodium, or potassium.

4. The method of claim 1, wherein the one or more amino acid(s) or derivative(s) thereof comprise one or more of lysine or leucine.

5. The method of claim 1, wherein the one or more antioxidant(s) comprise one or more of carnitine, taurine, or coenzyme Q10.

6. The method of claim 1, wherein the βHB comprises D-β-hydroxybutyrate.

7. The method of claim 1, wherein the βHB or acid thereof is selected and comprises a salt thereof, which comprises a calcium salt, a magnesium salt, a sodium salt, or a combination thereof.

8. The method of claim 1, wherein the therapeutically effective amount of βHB, the salt, or the metabolic precursor thereof is between about 0.1 gram and about 100 grams per day.

9. The method of claim 1, wherein a single serving of the composition provides about 0.5 g to about 20 g of βHB.

10. The method of claim 1, wherein the composition is administered in multiple daily doses for at least about 4 weeks.

11. The method of claim 10, wherein the composition is administered one to three times daily for about 4 weeks to about 8 weeks.

12. The method of claim 1, wherein administering the composition elevates blood βHB in the subject to between about 0.05 mM and about 1 mM.

13. The method of claim 1, wherein the depression comprises major depressive disorder (MDD).

14. The method of claim 1, which further comprises administering a therapeutically effective amount of an antidepressant agent, an anti-inflammatory agent, or both, to the subject.

15. The method of claim 14, wherein the antidepressant agent comprises a selective serotonin reuptake inhibitor (SSRI), a serotonin-norepinephrine reuptake inhibitor (SNRI), an atypical antidepressant, a tricyclic antidepressant, or a monoamine oxidase inhibitor (MAOI), or any combination thereof.

16. The method of claim 1, wherein inflammation is concurrently managed in the subject.

17. A method for the dietary management of depression in a human subject, which comprises:
orally administering to the subject, three times daily for at least about 4 weeks, a composition comprising D-β-hydroxybutyrate, or an acid thereof, provided as a calcium salt, a magnesium salt, or a combination thereof, together with a micronutrient component comprising one or more vitamin(s), one or more mineral nutrient(s), one or more amino acid(s) or derivative(s) thereof, one or more antioxidant(s), or a combination thereof; and
assessing depressive symptoms of the subject using a clinician-rated or self-reported depression scale.

18. The method of claim 17, wherein the clinician-rated scale comprises a Montgomery-Åsberg Depression Rating Scale (MADRS) and wherein the MADRS score is reduced by at least 5 points after about 8 weeks of administration of the composition, or the self-reported depression scale comprises a Quick Inventory of Depressive Symptomatology (QIDS) scale and wherein the QIDS score is reduced by at least 1.5 points after about 8 weeks of administration of the composition.

19. A composition for the dietary management of depression in a subject in need thereof, which comprises a therapeutically effective amount of β-hydroxybutyrate (βHB), a salt or acid thereof, or a metabolic precursor thereof and a micronutrient component comprising one or more vitamin(s), one or more mineral nutrient(s), one or more amino acid(s) or derivative(s) thereof, one or more antioxidant(s), or a combination thereof.

20. The composition of claim 19, formulated as a food product, a food supplement, or a medical food.

21. The composition of claim 19, wherein the βHB salt is selected and comprises D-β-hydroxybutyrate provided as a calcium salt, a magnesium salt, a sodium salt, or a combination thereof.

22. The composition of claim 19, packaged as unit-dose stick packs configured to be dissolved in water prior to ingestion.

23. The composition of claim 22, wherein each unit-dose stick comprises about 9 g of total formulation and provides about 5 g of βHB.

24. A composition comprising a therapeutically effective amount of β-hydroxybutyrate (βHB), a salt or acid thereof, or a metabolic precursor thereof, and a micronutrient component comprising one or more vitamin(s), one or more mineral nutrient(s), one or more amino acid(s) or derivative(s) thereof, one or more antioxidant(s), or a combination thereof for use in the dietary management of depression in a subject in need thereof.

25. The composition of claim 24, formulated as a food product, a food supplement, or a medical food.

26. The composition of claim 24, wherein the βHB salt comprises D-β-hydroxybutyrate provided as a calcium salt, a magnesium salt, a sodium salt, or a combination thereof.
